(19) United States
(12) Patent Application Publication
(10) Pub. No.: US 2025/0163037 A1
(43) Pub. Date: May 22, 2025

(54) SUBSTITUTED ISOXAZOLINE DERIVATIVES

(71) Applicant: BASF SE, Ludwigshafen (DE)

(72) Inventors: Nikolas Huwyler, Ludwigshafen (DE); Erik Gilberg, Koln (DE); Karsten Koerber, Ludwigshafen (DE)

(21) Appl. No.: 18/842,073

(22) PCT Filed: Feb. 22, 2023
(86) PCT No.: PCT/EP2023/054354

§ 371 Date: Aug. 28, 2024

(30) Foreign Application Priority Data
Mar. 2, 2022 (EP) 22159805.5
Mar. 2, 2022 (EP) 22159806.3

(51) Int. Cl. C07D 413/12; A01N 43/80; A01P 7/04

(52) U.S. Cl. CPC C07D 413/12; A01N 43/80; A01P 7/04

(57) ABSTRACT

The invention relates to isoxazoline compounds of formula (I) wherein the variables have the meanings as defined in the specification, to compositions comprising them, to active compound combinations comprising them, and to their use for protecting growing plants and animals from attack or infestation by invertebrate pests, furthermore, to seed comprising such compounds.

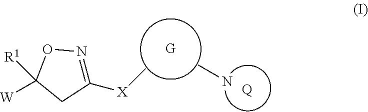

The invention relates to isoxazoline compounds of formula I

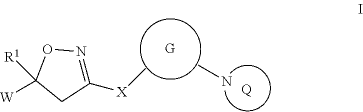

wherein
R¹ is C₁-C₂-haloalkyl;
W is phenyl, or pyridyl; wherein W is unsubstituted, partially or fully substituted with R²; R² is halogen, OR²¹, NR²²R²³, CN, NO₂, Si(CH₃)₃, SbF₅, C₁-C₄-alkyl, C₁-C₄-haloalkyl, C₃-C₆-cycloalkyl, C₃-C₆-halocycloalkyl, C₂-C₄-alkenyl, C₂-C₄-haloalkenyl, C₂-C₄-alkynyl, C₂-C₄-haloalkynyl, C₁-C₃-alkoxy, C₁-C₃-haloalkoxy, C₁-C₃-alkyl-S(O)m, C₁-C₃-haloalkyl-S(O)m, C₃-C₆-cycloalkyl-S(O)ₘ, C₁-C₃-alkoxy-C₁-C₄-alkyl, C₁-C₃-haloalkoxy-C₁-C₄-alkyl, C₁-C₃-alkyl-S(O)ₘ—C₁-C₄-alkyl, C₁-C₃-haloalkyl-S(O)ₘ—C₁-C₄-alkyl; which groups are unsubstituted, partially or fully substituted with R²¹¹;
   R²¹ H, C₁-C₆-alkyl, C₂-C₆-alkenyl, C₂-C₆-alkynyl, C₃-C₃-cycloalkyl, Si(C₁-C₄-alkyl)₃, C₁-C₃-alkyl-S(O)ₘ, C₃-C₆-cycloalkyl-S(O)ₘ, S(O)ₘR²⁴, which groups are unsubstituted, partially or fully substituted with R²¹¹;
   R²², R²³ H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, which are unsubstituted or partially or fully substituted with R²²¹; or
      C₁-C₆-alkyl-C(═O)OR²⁴, C₁-C₆-alkyl-C(=U)N(R^{25a})R^{25b}, C₁-C₆-alkyl-C(═NR²⁵)N(R^{25a})R^{25b},
      S(O)ₘR²⁴, S(O)ₘN(R^{25a})R^{25b}, C(=U)R²⁶, C(═O)OR²⁴, C(=U)N(R^{25a})R^{25b}, C(═S)SR²⁴, C(=NR²⁵)R²⁶;
      C₃-C₃-cycloalkyl, C₃-C₃-halocycloalkyl;
   R²² and R²³ form together with the nitrogen atom they are bonded to a 3-, 4-, 5-, or 6-membered fully unsaturated heterocycle, which heterocycle may additionally contain one heteroatom selected from N, O, and S(O)ₘ as ring members, and which heterocycle is unsubstituted or partially or fully substituted with R²²²; or
   R²² and R²³ together form a group ═C(R²⁶)₂, =S(O)ₘ(R²⁴)₂, =S(O)ₘR²⁴N(R^{25a})R^{25b};
U is O or S;
   R²⁴ is H, Si(C₁-C₄-alkyl)₃, C₁-C₆-alkyl, C₂-C₆-alkenyl, C₂-C₆-alkynyl, which are unsubstituted or partially or fully halogenated and/or substituted with C₃-C₄-cycloalkyl, C₁-C₄-alkoxy, C₁-C₄-haloalkoxy, S(O)ₘ—C₁-C₄-alkyl, and oxo;
      C₃-C₈-cycloalkyl which is unsubstituted or partially or fully halogenated and/or substituted with C₁-C₄-alkyl, C₃-C₄-cycloalkyl, C₁-C₄-alkoxy, C₁-C₄-haloalkoxy, S(O)ₘ—C₁-C₄-alkyl, and oxo;
      phenyl, benzyl, pyridyl and phenoxy, which are unsubstituted or partially or fully halogenated and/or substituted with C₁-C₆-alkyl, C₁-C₆-haloalkyl, C₁-C₆-alkoxy, C₁-C₆-haloalkoxy, C₁-C₆-alkylthio, C₁-C₆-haloalkylthio, and (C₁-C₆-alkoxy)carbonyl;
   R²⁵ is H, C₁-C₆-alkoxy, C₁-C₆-haloalkoxy, C(=U)R²⁶, C(═O)OR²⁴, C(═O)NH(C₁-C₄-alkyl), C(═O)N(C₁-C₄-alkyl)₂, S(O)ₘR²⁴, S(O)ₘ—C₁-C₄-alkyl, S(O)ₘ—C₁-C₄-haloalkyl, S(O)ₘ—C₃-C₆-cycloalkyl, Si(C₁-C₄-alkyl)₃,
      C₁-C₆-alkyl, C₃-C₆-alkenyl, C₃-C₆-alkynyl, which are unsubstituted or partially or fully halogenated and/or substituted with CN, C₁-C₄-alkoxy, C₁-C₄-haloalkoxy, S(O)ₘ—C₁-C₄-alkyl, S(O)ₘ—C₁-C₄-haloalkyl, C₃-C₆-cycloalkyl which is unsubstituted or substituted with 1 or 2 halogen and/or CN; phenyl, or 5-, 6-membered heteroaryl containing 1, 2 or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio; and oxo;
      C₃-C₈-cycloalkyl which is unsubstituted or partially or fully halogenated and/or substituted with CN, C₁-C₄-alkyl, C₁-C₄-alkoxy, C₁-C₄-haloalkyl, C₁-C₄-haloalkoxy, S(O)ₘ—C₁-C₄-alkyl, S(O)ₘ—C₁-C₄-haloalkyl, C(═O)NH(C₁-C₄-alkyl), C(═O)N(C₁-C₄-alkyl)₂, phenyl, or 5-, 6-membered heteroaryl containing 1, 2 or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio; phenyl, benzyl, pyridyl, phenoxy, which are unsubstituted or partially or fully halogenated and/or substituted with halogen, CN, NO₂, C₁-C₆-alkyl, C₁-C₆-haloalkyl, C₁—CO₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, and (C₁-C₆-alkoxy)carbonyl; and a 3-, 4-, 5- or 6-membered saturated, partially or fully unsaturated heterocycle comprising 1, 2 or 3 heteroatoms selected from N, O, and S(O)ₘ as ring members, where the heterocycle is optionally substituted with one or more R²²²;
   R^{25a} and R^{25b} have the meanings given for R²⁵; or
   R^{25a} and R^{25b} present on the same nitrogen atom may together form ═C(R²⁶)₂, ═S(O)ₘ(R²⁴)₂, or ═S(O)ₘR²⁴N(R^{25a})R^{25b}; or
   R^{25a} and R^{25b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo; or
   R^{25a} and R²⁵, together with the nitrogen atoms to which they are bound in the group C(═NR²S)N(R^{25a})R^{25b}, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo;
   R²⁶ is H, OH, SH, and C₁-C₆-alkyl, C₂-C₆-alkenyl, C₂-C₆-alkynyl, C₃-C₃-cycloalkyl, which are unsubstituted, partially or fully halogenated and/or substituted with 1, 2, or 3 groups CN, C₁-C₄-alkyl, C₃-C₄-cycloalkyl, C₁-C₄-haloalkyl, C₁-C₄-alkoxy, C₁-C₄-haloalkoxy, S(O)ₘ—C₁-C₄-alkyl, S(O)ₘ—C₁-C₄-haloalkyl, phenyl, or 3-, 4-, 5-, 6- or 7-membered saturated, partially or fully unsaturated heterocycle containing 1, 2 or 3 heteroatoms N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio; and oxo;
      C₁-C₆-alkoxy, C₁-C₆-haloalkoxy, S(O)ₘ—C₁-C₆-alkyl, S(O)ₘ—C₁-C₆-haloalkyl, Si(C₁-C₄-alkyl)₃, C(═O)N(R^{25a})R^{25b},
      phenyl, benzyl, phenoxy, a 3-, 4-, 5-, 6- or 7-membered saturated, partially or fully unsaturated heterocycle containing 1, 2 or 3 heteroatoms selected from N, O, and S(O)ₘ as ring members, which rings are unsubstituted or partially or fully halogenated and/or substituted with R²²²;
   R²¹¹ halogen, CN, C₁-C₄-alkoxy, C₁-C₄-haloalkoxy, C₃-C₄-alkenyloxy, C₃-C₄-haloalkenyloxy, C₃-C₄-alkynyloxy, C₃-C₄-haloalkynyloxy, C₁-C₄-alkyl-S(O)ₘ, C₁-C₄-haloalkyl-S(O)ₘ, C₃-C₄-alkenyl-S(O)ₘ, C₃-C₄-haloalkenyl-S(O)ₘ, C₃-C₄-alkynyl-S(O)ₘ, C₃-C₄-haloalkynyl-S(O)ₘ, and oxo;
      C₃-C₃-cycloalkyl, C₃-C₃-halocycloalkyl, C₃-C₃-cycloalkenyl, C₃-C₃-halocycloalkenyl;
   R²²¹ CN, NO₂, OH, SH, SCN, SF₅, Si(C₁-C₄-alkyl)₃, C₁-C₆-alkoxy, C₁-C₆-haloalkoxy, C₁-C₆-alkyl-S(O)ₘ, C₁-C₆-haloalkyl-S(O)ₘ, C(═O)N(R^{25a})R^{25b};
      C₃-C₃-cycloalkyl which is unsubstituted, partially or fully halogenated and/or partially or fully substituted with C₁-C₄-alkyl, C₃-C₄-cycloalkyl, C₁-C₄-alkoxy, C₁-C₄-haloalkoxy, and oxo; or
   two R²²¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
   R²²² is halogen, NO₂, CN, OH, SH, C₁-C₆-alkoxy, C₁-C₆-haloalkoxy, S(O)ₘ—C₁-C₄-alkyl, S(O)ₘ—C₁-C₄-haloalkyl, C₁-C₄-alkylcarbonyl, C₁-C₄-haloalkylcarbonyl, C₁-C₄-alkoxycarbonyl, C₁-C₄-haloalkoxycarbonyl, N(R^{25a})R^{25b}, C(═O)NR^{25a}R^{25b}, Si(C₁-C₄-alkyl)₃;
      C₁-C₄-alkyl, C₂-C₆-alkenyl, C₂-C₆-alkynyl, which are unsubstituted or partially or fully halogenated and/or substituted with CN, C₃-C₄-cycloalkyl, C₁-C₄-alkoxy, C₁-C₄-haloalkoxy, S(O)ₘ—C₁-C₄-alkyl, and oxo;
      C₃-C₃-cycloalkyl which is unsubstituted or partially or fully halogenated and/or substituted with CN, C₁-C₄-alkyl, C₃-C₄-cycloalkyl, C₁-C₄-alkoxy, C₁-C₄-haloalkyl, C₁-C₄-haloalkoxy, S(O)ₘ—C₁-C₄-alkyl, and oxo; or
   two R²²² present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl; or
   two R²²² on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from N, O, and S(O)ₘ as ring members, and wherein the ring is optionally substituted with one or more groups C₁-C₄-alkyl, C₁-C₄-haloalkyl, C₁-C₄-alkoxy, and/or C₁-C₄-haloalkoxy;
   m is 0, 1, or 2;
X is NR³, or O;
R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OR²¹, C₁-C₆-alkylthio, C₁-C₆-haloalkylthio, which are unsubstituted or partially or fully substituted with R³¹; or
   C₁-C₆-alkyl-C(═O)OR²⁴, C₁-C₆-alkyl-C(=U)N(R^{25a})R^{25b}, C₁-C₆-alkyl-C(═NR²⁵)N(R^{25a})R^{25b}, C₁-C₆-alkyl-OC(═O)OR²⁴, N(R^{25a})R^{25b}, S(O)ₘR²⁴, S(O)ₘN(R^{25a})R^{25b}, C(=U)R²⁶, C(═O)OR²⁴, C(=U)N(R^{25a})R^{25b}, C(═S)SR²⁴, C(═NR²⁵)R²⁶;
   C₃-C₃-cycloalkyl, C₃-C₃-halocycloalkyl, phenyl, a 3-, 4-, 5-, 6- or 7-membered saturated or partially unsaturated heterocycle comprising 1, 2, 3 or 4 heteroatoms selected from N, O, and S(O)ₘ as ring members, or a 5- or 6-membered hetaryl comprising 1, 2, 3 or 4 heteroatoms selected from N, O, and S(O)ₘ as ring members, which rings are unsubstituted or partially or fully substituted with R³²;
   R³¹ halogen, CN, NO₂, OH, SH, SCN, SF₅, Si(C₁-C₄-alkyl)₃, N(R^{25a})R^{25b}, C₁-C₆-alkoxy, C₁-C₆-haloalkoxy, C₁-C₆-alkyl-S(O)ₘ, C₁-C₆-haloalkyl-S(O)ₘ, C(═O)N(R^{25a})R^{25b};
      C₃-C₃-cycloalkyl which is unsubstituted, partially or fully halogenated and/or partially or fully substituted with CN, C₁-C₄-alkyl, C₃-C₄-cycloalkyl, C₁-C₄-alkoxy, C₁-C₄-haloalkyl, C₁-C₄-haloalkoxy, and oxo;
      S(O)ₘR²⁴, S(O)ₘN(R^{25a})R^{25b}, C(=U)R²⁶, C(═O)OR²⁴, C(=U)N(R^{25a})R^{25b}, C(═S)SR²⁴, C(═NR²⁵)R²⁶;
      phenyl, benzyl, phenoxy, or 3-, 4-, 5-, 6- or 7-membered saturated, partially or fully unsaturated heterocycle containing 1, 2, or 3 heteroatoms selected from N, O, S(O)ₘ as ring members, wherein the rings are unsubstituted or partially or fully substituted with R²²²; or
      two R³¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
   R³² is a group as defined in R³¹ or selected from C₁-C₆-alkyl, C₂-C₆-alkenyl, and C₂-C₆-alkynyl, which groups are unsubstituted, partially or fully halogenated and/or substituted with one or two CN, C₃-C₄-cycloalkyl, C₁-C₄-alkoxy, C₁-C₄-haloalkoxy, or oxo;
G is phenyl, or 6-membered heteroaryl comprising as ring members 1 or 2 N-atoms; wherein G is unsubstituted, partially, or fully substituted with R²;
Q is an N-bound 5-membered heteroaryl comprising as ring members 1, 2, or 3 N-atoms; wherein Q is unsubstituted, partially, or fully substituted with R⁴;
R⁴ is halogen, NO₂, CN, OH, SH, SbF₅, C₁-C₆-alkoxy, C₁-C₆-haloalkoxy, C₁-C₄-alkylcarbonyl, C₁-C₄-haloalkylcarbonyl, C₁-C₄-alkoxycarbonyl, C₁-C₄-haloalkoxycarbonyl, Si(C₁-C₄-alkyl)₃; OR²¹, S(O)ₘ—C₁-C₄-alkyl, S(O)ₘ—C₁-C₄-haloalkyl, N(R^{25a})R^{25b}, S(O)ₘR²⁴, S(O)ₘN(R^{25a})R^{25b}C(═NR²⁵)R²⁶, C(=U)R²⁶, C(═O)OR²⁴, C(=U)N(R^{25a})R^{25b}, C(═S)SR²⁴;
   C₁-C₄-alkyl, C₂-C₆-alkenyl, C₂-C₆-alkynyl, which are unsubstituted or partially or fully halogenated and/or substituted with CN, C₃-C₆-cycloalkyl, C₁-C₄-alkoxy, C₁-C₄-haloalkoxy, S(O)ₘ—C₁-C₄-alkyl, S(O)ₘ—C₁-C₄-haloalkyl, N(R^{25a})R^{25b}, N(=O)(R^{25a})R^{25b}, and oxo;
   C₃-C₈-cycloalkyl which is unsubstituted or partially or fully halogenated and/or substituted with CN, C₁-C₄-alkoxy, C₁-C₄-haloalkoxy, S(O)ₘ—C₁-C₄-alkyl, S(O)ₘ—C₁-C₄-alkyl, N(R^{25a})R^{25b}, N(=O)(R^{25a})R^{25b}; and oxo;
   phenyl, benzyl, phenoxy, or 3-, 4-, 5-, 6- or 7-membered saturated, partially or fully unsaturated heterocycle containing 1, 2, or 3 heteroatoms selected from N, O, S(O)ₘ as ring members, wherein the rings are unsubstituted or partially or fully substituted with R²²²;
the N-oxides, stereoisomers and agriculturally or veterinarily acceptable salts thereof.

The invention also provides an agricultural composition comprising at least one compound of formula I, a stereoisomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The present invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasiticidally effective amount of a compound of formula I or a veterinarily acceptable salt thereof. Bringing the animal in contact with the compound I, its salt or the veterinary composition of the invention means applying or administering it to the animal.

WO2007/075459, WO2007/070606, WO2008/150393, WO2008/019760, WO2009/051956, WO2010/020522, WO2011/073444, EP1997813, and WO2022/171472 describe structurally closely related active compounds. These compounds are mentioned to be useful for combating invertebrate pests.

Nevertheless, there remains a need for highly effective and versatile agents for combating invertebrate pests. It is therefore an object of the present invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

Compounds of formula I with X being NR³ (formula 1.1) can be prepared by reaction of a compound of formula IIA.1 with a compound of formula IIIA, wherein XH is a halogen, preferably a bromide or iodide, in a Buchwald-Hartwig reaction [cf. PCT/EP2022/052181; WO2010/135360].

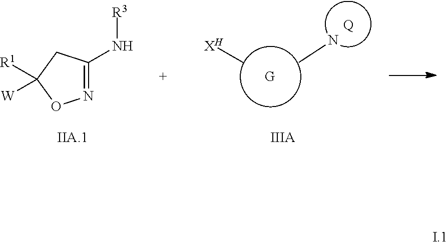

This transformation is usually carried out at temperatures from 20° C. to 180° C., preferably from 60° C. to 100° C., in an inert solvent, in the presence of a base and a palladium catalyst such as tris(dibenzylideneacetone)dipalladium(0)/9,9-dimethyl-4,5-bis(diphenylphosphino)xanthene (Pd₂(dba)₃/XantPhos), [2-(di-tert-butylphosphino)-2′,4′,6′-triisopropyl-1,1′-biphenyl][2-(2-aminoethyl)-phenyl)]palladium(II) chloride [cf. WO2017/069980]. Suitable solvents are ethers such as 1,4-dioxane, THF; or alcohols such as butanol, tert-amyl alcohol; or polar aprotic solvents such as dimethylformamide (DMF), dimethylacetamide (DMA), N-methyl-2-pyrrolidon (NMP), dimethyl sulfoxide (DMSO). Preferably, 1,4-dioxane is employed. It is also possible to use mixtures of the solvents mentioned. Suitable bases are, in general, inorganic compounds, like alkali metal and alkaline earth metal carbonates, such as e.g. Li₂CO₃, K₂CO₃, Cs₂CO₃, CaCO₃; or organic compounds, such as alkali metal alcoholates, such as lithium tert-butylate, sodium tert-butylate, potassium tert-butylate. Particular preference is given to Cs₂CO₃. The bases are generally employed in equimolar amounts; however, they can also be used in in excess.

The starting materials are generally reacted with one another in equimolar amounts. In terms of yield, it may be advantageous to employ an excess of IIA.1, based on IIIA.

Compounds of formula IIA.1 are known from the literature [cf. PCT/EP2022/052181] and can be prepared as described therein.

Alternatively, compounds of formula I, can also be prepared by reaction of a compound of formula IIB, wherein X^{H} is a halogen, preferably a bromide or chloride, with a compound of formula IIIB in a base-promoted ipso-substitution reaction [cf. PCT/EP2022/052181].

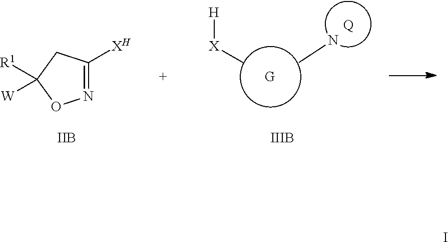

This transformation is usually carried out at temperatures of from 25° C. to 200° C., preferably from 60° C. to 150° C., in an inert solvent, in the presence of a base. Suitable solvents are DMF, DMA, NMP, DMSO, and the like; particular preference is given to NMP, and DMF. It is also possible to use mixtures of the solvents mentioned. Suitable bases are, in general, inorganic compounds, like alkali metal and alkaline earth metal hydroxides, such as e.g. LiOH, NaOH, KOH, Ca(OH)₂; or alkali metal and alkaline earth metal hydrides, such as e.g. LiH, NaH, KH, CaH₂; or alkali metal and alkaline earth metal carbonates, such as e.g. Li₂CO₃, K₂CO₃, CaCO₃; or organic bases, like N-containing heteroaromatics such as pyridine, 2,6-lutidine. The bases are generally employed in equimolar amounts; however, they can also be used in in excess or, if appropriate, as solvent.

Compounds of formula IIB can be prepared as described in the literature [cf. PCT/EP2022/052181].

In addition, compounds of formula 1.1 can be prepared by reaction of a compound of formula IIB, wherein X^{H} is a halogen, preferably a bromide or chloride, with an amine of formula IIIB.1 in an acid-promoted ipso-substitution reaction [cf. PCT/EP2022/052181].

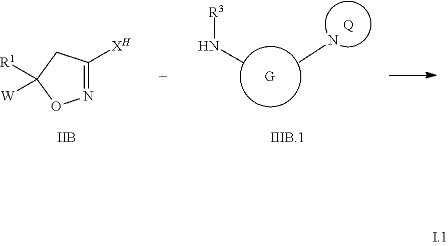

This transformation is usually carried out at temperatures of from 25° C. to 200° C., preferably from 60° C. to 150° C., in an inert solvent, in the presence of an acid. Suitable solvents are alcohols such as 2,4-dimethylpentan-3-ol, n-butanol, sec-butanol, tert-butanol; or aromatic hydrocarbons such as e.g. toluene, o-, m-, p-xylene, chlorobenzene, dichlorobenzene; or polar aprotic solvents such as e.g. DMSO, DMF, DMA, NMP; preferably alcohols such as 2,4-dimethylpentan-3-ol are employed. It is also possible to use mixtures of the solvents mentioned. Suitable acids and acidic catalysts are, in general, inorganic acids such as e.g. HCl, HBr, H₂SO₄, HCIO₄; or organic acids such as e.g. toluenesulfonic acid, benzenesulfonic acid, camphor sulfonic acid, trifluoromethanesulfonic acid, trifluoroacetic acid. The acids are generally employed in catalytic amounts; however, they can also be used in equimolar amounts, in excess or, if appropriate, as solvent. The starting materials are generally reacted with one another in equimolar amounts. In terms of yield, it may be advantageous to employ an excess of IIIB.1, based on IIB.

Furthermore, compounds of formula I can also be prepared by reaction of an olefin of formula VI with an in-situ formed compound of formula V, which in turn is derived from the reaction of a compound of formula IIIB with a dihaloaldoxime of formula IV, in a 1,3-dipolar cycloaddition reaction [cf. PCT/EP2022/052181].

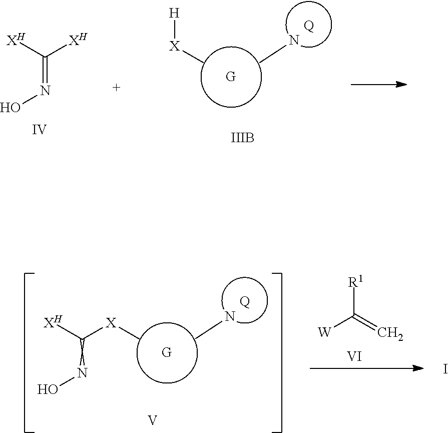

This transformation is usually carried out at temperatures from −78° C. to 80° C., preferably from −78° C. to 25° C., in the presence of a base. Suitable solvents are ethers such as e.g. THF, 1,4-dioxane, Et₂O, tert-butylmethylether; or esters such as e.g. EtOAc (acetate=OAc); or aromatic hydrocarbons such as e.g. benzene, toluene, o-, m-, and p-xylene, chlorobenzene, dichlorobenzene; or halogenated hydrocarbons such as, e.g. CH₂Cl₂, CHCl₃, 1,2-dichloroethane. It is also possible to use mixtures of the solvents mentioned. Suitable bases are, in general, organic bases like tertiary amines, such as e.g. triethylamine, diisopropylethylamine; or N-containing heteroaromatics such as e.g. pyridine, collidine, 2,6-lutidine, 4-dimethylaminopyridine; or inorganic compounds, like alkali metal and alkaline earth metal carbonates, such as e.g. Li₂CO₃, K₂CO₃, CaCO₃; or alkali metal bicarbonates, such as e.g. NaHCO₃, KHCO₃; or alkali metal phosphates such as e.g. K₃PO₄. The bases are generally employed in equimolar amounts; however, they can also be used in excess or, if appropriate, as solvent.

The starting materials are generally reacted with one another in equimolar amounts. In terms of yield, it may be advantageous to employ an excess of IV and IIIB, based on VI.

Compounds of formula IV are commercially available, and the preparation of compounds of formula VI are described in the literature [cf. e.g. PCT/EP2022/052181].

Compounds of formula IIIA, in turn, can be prepared by reacting a compound of formula VII, wherein X^{LG} is a leaving group, preferably a fluorine or chlorine atom, with an N-heterocyclic compound of formula VIII in a base-promoted nucleophilic aromatic substitution reaction.

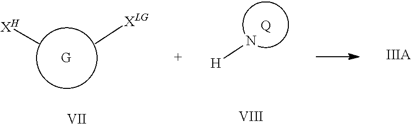

This reaction is usually carried out at temperatures from −20° C. to 180° C., preferably from 25° C. to 120° C., in an inert solvent, and in the presence of a base [cf. WO 2010/100189]. Suitable solvents are e.g. DMSO, DMF, DMA, NMP; or nitriles such as acetonitrile, propionitrile; or ethers such as 1,4-dioxane, THF; or aromatic hydrocarbons such as toluene, o-, m-, p-xylene; or alcohols such as methanol, ethanol, n-propanol, isopropanol, n-butanol, tert-butanol; or water. It is also possible to use mixtures of the solvents mentioned. Suitable bases are, in general, inorganic compounds, like alkali metal and alkaline earth metal carbonates, such as e.g. Li₂CO₃, K₂CO₃, Cs₂CO₃, CaCO₃; or alkali metal and alkaline earth metal hydrides, such as e.g. LiH, NaH, KH, CaH₂; or organic bases, like tertiary amines, such as e.g. trimethylamine, triethylamine, diisopropylethylamine, N-methylpiperidine; or N-containing aromatics such as e.g. pyridine, collidine, 2,6-lutidine, 4-dimethylaminopyridine; or alkali metal amides such as e.g. LiN(iPr)₂, LiN(SiMe₃)₂, NaN(SiMe₃)₃, KN(SiMe₃)₂; or alkali metal alcoholates such as potassium tertbutoxide. The bases are generally employed in equimolar amounts; however, they can also be used in in excess or, if appropriate, as solvent. The starting materials are generally reacted with one another in equimolar amounts. In terms of yield, it may be advantageous to employ an excess of VIII, based on VII.

Alternatively, it is also possible to prepare compounds of formula IIIA by reacting a compound of formula VII, wherein X^{LG} preferably is iodide, bromide, or triflate, with an N-heterocyclic compound of formula VIII in a palladium-catalyzed (i.e. Buchwald-Hartwig reaction) cross-coupling reaction.

Buchwald-Hartwig reactions are usually carried out at temperatures from 25° C. to 200° C., preferably from 50° C. to 150° C., in an inert solvent, and in the presence of a palladium catalyst and a base [cf. WO2016/168059]. Suitable solvents are e.g. aromatic hydrocarbons such as toluene, o-, m-, and p-xylene; or ethers such as 1,4-dioxane, and THF; or nitriles such as acetonitrile, and propionitrile; or polar aprotic solvents such as DMSO, DMF, DMA, NMP. Suitable palladium catalysts are e.g. Pd(OAc)₂/PPh₃, Pd(OAc)₂/2,2′-bis(diphenylphosphino)-1,1′-binaphthyl (BINAP), Pd(OAc)₂/4,5-bis(diphenylphosphino)-9,9-dimethylxanthene (XantPhos), dichloro[9,9-dimethyl-4,5-bis(diphenylphosphino)xanthene]palladium(II), [1,1′-bis(diphenylphosphino)ferrocene]dichloropalladium(II), (1,3-bis(diphenylphosphino)propane)palladium(II) chloride, transbis(acetato)bis[o-(di-o-tolylphosphino)benzyl]dipalladium(II), [2-(di-tert-butylphosphino)-2′,4′,6′-triisopropyl-1,1′-biphenyl][2-(2-aminoethyl)-phenyl)]palladium(II) chloride. Suitable bases are, in general, inorganic compounds, like alkali metal and alkaline earth metal carbonates, such as e.g. Li₂CO₃, K₂CO₃, Cs₂CO₃, CaCO₃; or alkali metal phosphates such as e.g. K₃PO₄; or organic bases, like tertiary amines, such as e.g. triethylamine, diisopropylethylamine, N-methylpiperidine, N-methyl-N,N-dicyclohexylamine, and 1,4-diaza-bicyclo[2.2.2]octane; or amidines such as 1,8-diazabicyclo[5.4.0]undec-7-en; or alkali metal alcoholates such as sodium tert-butoxide; or alkali metal amides such as lithium bis(trimethylsilyl)amide (LiHMDS). The bases are generally employed in equimolar amounts; however, they can also be used in in excess or, if appropriate, as solvent.

Furthermore, it is also possible to prepare compounds of formula IIIA by reacting a compound of formula VII, wherein X^{LG} preferably is iodide, bromide, B(OH)₂, B(alkyl)₂, or B(O-alkyl)₂ with an N-heterocyclic compound of formula VIII in a copper-promoted (i.e. Ullmann or Chan-Lam-Evans reaction, resp.) cross-coupling reaction.

The copper-promoted reactions are usually carried out at temperatures from 25° C. to 200° C., preferably from 50° C. to 150° C., in an inert solvent, and in the presence of a copper catalyst and a base. Suitable solvents are e.g. N-containing aromatics such as pyridine, quinoline, and the like; or polar aprotic solvents such as DMSO, DMF, DMA, NMP; or ethers such as 1,4-dioxane, bis(2-methoxyethyl)ether; or aromatic hydrocarbons such as toluene, o-, m-, p-xylene; or nitriles such as acetonitrile, propionitrile; or alcohols such as methanol, isopropanol, tert-butanol; and it is also possible to use mixtures of the solvents mentioned. Suitable copper catalysts are e.g. CuI, CuBr, CuCl, Cu₂O, Cu(OAc)₂. If desired, it is also possible to employ an amine- or amide-based ligand in the reaction such as e.g. pyridine, 2,6-lutidine, 4-(dimethylamino)pyridine, quinoline, 1,10-phenantroline; or N,N,N′,N′-tetramethylethane-1,2-diamine, N,N-dimethylglycine. Suitable bases are, in general, organic bases, such as e.g. pyridine, 2,6-lutidine; or tertiary amines, such as triethylamine; or inorganic compounds, like alkali metal and alkaline earth metal carbonates, such as e.g. Li₂CO₃, K₂CO₃, Cs₂CO₃, CaCO₃; or alkali metal phosphates such as K₃PO₄. The bases are generally employed in equimolar amounts; however, they can also be used in in excess or, if appropriate, as solvent.

The starting materials are generally reacted with one another in equimolar amounts. In terms of yield, it may be advantageous to employ an excess of VIII, based on VII.

Aromatic compounds of formula VII and N-heterocyclic compounds of formula VIII are, in general, commercially available or known in the literature.

Compounds of formula IIIB.1, wherein X is NR³, can be prepared by reacting a compound of formula IIIA, wherein X^{H} is a halogen, preferably fluoride or chloride, with an amine R³NH₂ in a base-promoted nucleophilic aromatic substitution reaction under the same conditions as described above for the synthesis of IIIA from compounds of formula VII and VIII.

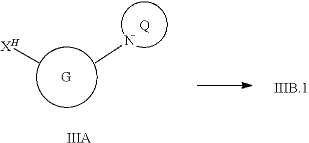

Alternatively, Compounds of formula IIIB.1, wherein X is NR³, can be prepared by reacting a compound of formula IIIA, wherein X^{H} is a halogen, preferably bromide or iodide, with an amine R³NH₂ in a Buchwald-Hartwig or Ullmann cross-coupling reaction, resp., under the same conditions as described above for the synthesis of IIIA from compounds of formula VII and VIII.

Furthermore, compounds of formula IIIB.1a, wherein X is NH, can be prepared from compounds of formula IX via reduction of the NO₂-group with SnCl₂, iron, or zinc, respectively.

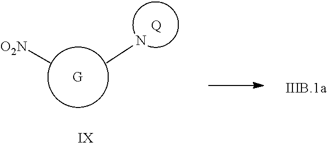

These reactions are usually carried out at temperatures from 25° C. to 180° C., preferably from 25° C. to 80° C., in an inert solvent, and in the presence of an acid. Suitable solvents are e.g. alcohols such as methanol, ethanol, isopropanol, n-butanol; or esters such as e.g. EtOAc; or ethers such as e.g. THF; or H₂O; and it is also possible to use mixtures of the solvents mentioned.

Suitable acids are, in general, inorganic acids like mineral acids such as e.g. HCl; or ammonium salts such as e.g. NH₄Cl; or organic acids like carboxylic acids such as e.g. AcOH. The acids are generally employed in equimolar amounts; however, they can also be used in in excess or, if appropriate, as solvent. The starting materials are generally reacted with one another in equimolar amounts. In terms of yield, it may be advantageous to employ an excess of SnCl₂, iron, or zinc, resp., based on IX.

In addition, compounds of formula IIIB.1a, wherein X is NH, can also be prepared from compounds of formula IX via reduction of the NO₂-group with a Pd on activated carbon catalyst.

This reaction is usually carried out at temperatures from 0° C. to 100° C., preferably from 25° C. to 50° C., in an inert solvent, and under a 1-10 bar atmosphere of H₂. Suitable solvents are e.g. alcohols such as methanol, ethanol, isopropanol, n-butanol; or esters such as e.g. EtOAc; or ethers such as e.g. THF; or H₂O; and it is also possible to use mixtures of the solvents mentioned.

Compounds of formula IIIB.2, wherein X is O, can be prepared by reacting a compound of formula X, wherein X^{LG} is a leaving group, preferably iodide, bromide, triflate, B(OH)₂, B(alkyl)₂, or B(O-alkyl)₂, with an N-heterocyclic compound of formula VIII in a Buchwald-Hartwig, Ullmann, or Chan-Lam-Evans reaction, resp., under the same conditions as described above for the synthesis of IIIA from compounds of formula VII and VIII.

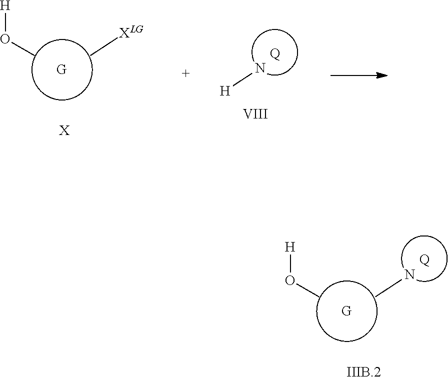

In turn, compounds of formula IX can be prepared by reacting a compound of formula XI, wherein X^{LG} is a leaving group, preferably F or Cl, with an N-heterocyclic compound of formula VIII in a base-promoted nucleophilic aromatic substitution reaction under the conditions as described above for the synthesis of IIIA from compounds of formula VII and VIII.

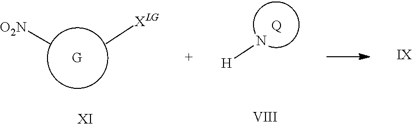

Alternatively, compounds of formula IX can be prepared by reacting a compound of formula XI, wherein X^{LG} is a leaving group, preferably iodide, bromide, triflate, B(OH)₂, B(alkyl)₂, or B(O-alkyl)₂, with an N-heterocyclic compound of formula VIII in a Buchwald-Hartwig, Ullmann, or Chan-Lam-Evans reaction, resp., under the conditions as described above for the synthesis of IIIA from compounds of formula VII and VIII.

Compounds of formula XI are commercially available or known in the literature.

If desired, compounds of formula 1.1, wherein R³ is not H, can also be prepared by alkylation or acylation of the corresponding H-compound (R³=H), analogously as previously described in the literature [cf. PCT/EP2022/052181].

The starting materials required for preparing the compounds I are commercially available or known from the literature [cf. PCT/EP2022/052181] or can be prepared in accordance with the literature cited.

Compounds of formula I.S, that is compounds of formula I wherein U is S, can be prepared by re-acting the corresponding oxocompound (U is O) with Lawesson's reagent (CAS 19172-47-5), see, e.g. Jesberger et al. Synthesis, 2003, 1929-1958 and references therein. Solvents such as HMPA or THF at an elevated temperature such as 60° C. to 100° C. can be used. Preferred reaction conditions are in THF at 65° C.

The reaction mixtures are worked up in a customary manner, for example by mixing with water, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

However, if the synthesis yields mixtures of isomers, a separation is generally not necessarily required since in some cases the individual isomers can be interconverted during work-up for use or during application (e.g. under the action of light, acids or bases). Such conversions may also take place after use, e.g. in the treatment of plants in the treated plant, or in the pest to be controlled.

In a preferred embodiment, the compounds I are present in form of a mixture of compounds I.A and I.B, wherein compound I.A with S-configuration in the isoxazoline ring is present in an amount of more than 50% by weight, in particular of at least 70% by weight, more particularly of at least 85% by weight, specifically of at least 90% by weight, based on the total weight of compounds I.A and I.B.

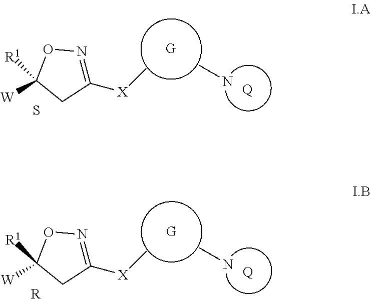

In one particularly preferred embodiment of the invention, the method comprises the step of contacting the plant, parts of it, its propagation material, the pests, their food supply, habitat or breeding grounds a pesticidally effective amount of a compound of formula l.A.

Compounds of formula IA, and I.B, resp., can be obtained in enantiopure form by known separation methods, preferably by chiral chromatography. This is preferably applied to intermediate compounds of formula IA and IIB, or to compounds of formula I.

The organic moieties groups mentioned in the above definitions of the variables are—like the term halogen—collective terms for individual listings of the individual group members. The prefix Cn-Cm indicates in each case the possible number of carbon atoms in the group.

The term “partially or fully substituted” by a radical means that in general the group is substituted with same or different radicals.

The term “halogen” denotes in each case fluorine, bromine, chlorine, or iodine, in particular fluo-rine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl (n-Pr), iso-propyl, n-butyl, 2-butyl, iso-butyl, tert-butyl, n-pentyl, 1-me-thylbutyl, 2 methylbutyl, 3 methylbutyl, 2,2-di-methylpropyl, 1 ethylpropyl, n-hexyl, 1,1-dimethyl-propyl, 1.2-dimethylpropyl, 1-methylpentyl, 2-methylpentyl, 3-methylpentyl, 4-methylpentyl, 1,1-dimethyl-butyl, 1,2-dimethylbutyl, 1,3-dimethyl-butyl, 2,2-dimethylbutyl, 2,3-dimethylbutyl, 3,3-dimethyl-butyl, 1-ethylbutyl, 2-ethylbutyl, 1,1,2-trimethylpropyl, 1,2,2-trimethylpropyl, 1-ethyl-1-methyl-propyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-halo-alkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2 difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-prop-oxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoromethoxy, 1 fluoroethoxy, 2-fluoroethoxy, 2,2 difluoroethoxy, 2,2,2-trifluoroethoxy, 2-chloro-2-fluoroethoxy, 2-chloro-2,2-difluoro-ethoxy, 2,2dichloro-2-fluorethoxy, 2,2,2-trichloroethoxy, penta-fluoroethoxy and the like.

The term “alkylthio “(alkylsulfanyl: alkyl-S—)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: C₁-C₆-alkyl-S(O)-), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (alkyl-S(O)₂—) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the car-bon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2 propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radi-cal having usually 2 to 10, frequently 2 to 6, preferably 2 to 4 carbon atoms, e.g. ethynyl, propar-gyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl (c-C₃H₅), cyclobutyl, cyclopentyl, cyclohexyl, cycloheptyl, cyclooctyl, cyclononyl and cyclodecyl or cyclopropyl, cyclobutyl, cyclopentyl and cyclo-hexyl.

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “halocycloalkenyl” as used herein and in the halocycloalkenyl moieties of halocycloalkenyloxy and halocycloalkenylthio denotes in each case a monocyclic singly unsaturated nonaromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen1-yl and 3,3-dichlorocyclopropen-1-yl.

The term “cycloalkenylalkyl” refers to a cycloalkenyl group as defined above which is bonded via an alkyl group, such as a C₁-C₆-alkyl group or a C₁-C₄-alkyl group, in particular a methyl group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered monocyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 3- to 6-membered, in particular 6-membered monocyclic heterocyclic non-aromatic radicals. The heterocyclic non-aromatic radicals usually comprise 1, 2, 3, 4 or 5, preferably 1, 2 or 3 hetero-atoms selected from N, O and S as ring members, wherein S-atoms as ring members may be present as S, SO or SO₂. Examples of 5- or 6-membered heterocyclic radicals comprise satu-rated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxo-thiolanyl, dihydrothienyl, S-oxodihydrothienyl, S-dioxodihydro-thienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothio-pyranyl, Soxo,dihydrothiopyranyl, S-dioxodihydrothiopyranyl, tetrahydrothiopyranyl, S-oxotetra-hydrothiopyranyl, S-dioxotetrahydrothiopyranyl, morpholinyl, thiomorpholinyl, S-oxothio-morpho-linyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-only, and the like.

The term “hetaryl” includes monocyclic 5- or 6-membered heteroaromatic radicals comprising as ring members 1, 2, 3 or 4 heteroatoms selected from N, O and S. N- or S-containing hetaryl groups may be present as positively charged onium, and form together with a neighbouring atom a mesoionic entity. Examples of 5- or 6 membered heteroaromatic radicals include pyridyl, i.e. 2-, 3-, or 4 pyridyl, pyrimidinyl, i.e. 2, 4- or 5-pyrimidinyl, pyrazinyl, pyridazinyl, i.e. 3- or 4 pyridazinyl, thienyl, i.e. 2- or 3-thienyl, furyl, i.e. 2- or 3-furyl, pyrrolyl, i.e. 2- or 3 pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5 isothiazolyl, pyrazolyl, i.e. 1-, 3-, 4- or 5-pyrazolyl, i.e. 1-, 2-, 4- or 5-imidazolyl, oxadiazolyl, e.g. 2- or 5 [1,3,4]oxadiazolyl, 4- or 5-(1,2,3-oxa-diazol)yl, 3- or 5-(1,2,4-oxadiazol)yl, 2- or 5 (1,3,4-thiadiazol)yl, thiadiazolyl, e.g. 2- or 5-(1,3,4-thia-diazol)yl, 4- or 5 (1,2,3 thiadiazol)yl, 3- or 5-(1,2,4-thiadiazol)yl, triazolyl, e.g. 1H-, 2H- or 3H 1,2,3 triazol-4-yl, 2H-triazol-3-yl, 1H-, 2H-, or 4H-1,2,4-triazolyl and tetrazolyl, i.e. 1H- or 2H tetrazolyl. The term “hetaryl” also includes bicyclic 8 to 10-membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzo-thienyl, indolyl, ind-azolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2 d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocyclylalkyl” and “hetarylalkyl” refer to heterocyclyl or hetaryl, respectively, as defined above which are bonded via a C₁-C₆-alkyl group or a C₁-C₄-alkyl group, in particular a methyl group (=heterocyclylmethyl or hetarylmethyl, respectively), to the remainder of the molecule.

The term “arylalkyl” and “phenylalkyl” refer to aryl as defined above and phenyl, respectively, which are bonded via C₁-C₆-alkyl group or a C₁-C₄-alkyl group, in particular a methyl group (=arylmethyl or phenylmethyl), to the remainder of the molecule, examples including benzyl, 1-phenylethyl, 2-phenylethyl, 2-phenoxyethyl etc.

The terms “alkylene”, “cycloalkylene”, “heterocycloalkylene”, “alkenylene”, “cycloalkenylene”, “heterocycloalkenylene” and “alkynylene” refer to alkyl, cycloalkyl, heterocycloalkyl, alkenyl, cycloalkenyl, heterocycloalkenyl and alkynyl as defined above, respectively, which are bonded to the remainder of the molecule, via two atoms, preferably via two carbon atoms, of the respective group, so that they represent a linker between two moieties of the molecule.

In a particular embodiment, the variables of the compounds of the formula I have the following meanings, these meanings, both on their own and in combination with one another, being particular embodiments of the compounds of the formula 1.

Embodiments and preferred compounds of the invention for use in pesticidal methods and for insecticidal application purposes are outlined in the following paragraphs.

With respect to the variables, the particularly preferred embodiments of the intermediates correspond to those of the compounds of the formula 1.

In one embodiment W is a group WA,

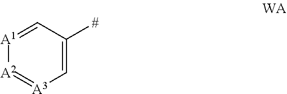

wherein
A¹, A², A³ are N or CR², provided that at least two are CR²;
R² are independently from each other preferably H, halogen, halomethyl, halomethoxy, or halomethyl-S(O)ₘ.

W is preferably a group WP:

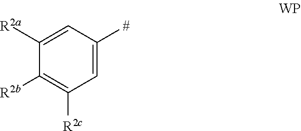

wherein R^{2a}, R^{2b}, and R^{2c} are a group R².

R^{2a} is preferably selected from F, Cl, Br, CF₃, SCF₃, and OCF₃.

R^{2b} and R^{2c} are independently preferably selected from H, F, Cl, Br, CF₃, SCF₃, and OCF₃.

Particularly preferred is each one of the following combinations of R^{2a}, R^{2b} and R^{2c} wherein each line of Table W denotes a substitution pattern of the phenyl ring (“WP”) bearing the R^{2a}, R^{2b} and R^{2c} moieties.

TABLE W

| No. | R^{2a} | R^{2b} | R^{2c} |
|---|---|---|---|
| W-1 | F | F | H |
| W-2 | F | H | F |
| W-3 | F | F | F |
| W-4 | F | Cl | F |
| W-5 | F | Br | F |
| W-6 | F | H | Cl |
| W-7 | F | H | Br |
| W-8 | Cl | F | H |
| W-9 | Cl | H | Cl |
| W-10 | Cl | Cl | Cl |
| W-11 | Cl | F | Cl |
| W-12 | Cl | Br | Cl |
| W-13 | Cl | H | Br |
| W-14 | Br | F | H |
| W-15 | Br | H | Br |
| W-16 | Br | F | Br |
| W-17 | Br | Cl | Br |
| W-18 | CF₃ | H | H |
| W-19 | CF₃ | H | F |
| W-20 | CF₃ | H | Cl |
| W-21 | CF₃ | H | Br |
| W-22 | CF₃ | H | CF₃ |
| W-23 | CF₃ | F | F |
| W-24 | CF₃ | F | Cl |
| W-25 | CF₃ | Cl | Cl |
| W-26 | CF₃ | F | H |
| W-27 | OCF₃ | H | F |
| W-28 | OCF₃ | H | Cl |
| W-29 | OCF₃ | F | H |
| W-30 | OCF₃ | H | CF₃ |
| W-31 | OCF₃ | H | H |
| W-32 | Cl | OCF₃ | H |
| W-33 | SCF₃ | H | Cl |

Groups W-8, W-9, and W-11 are more preferred patterns in formula I compounds. W-8 and W-11 are particularly preferred.

Another embodiment of W is a 6-membered hetaryl, such as thiophene, pyrazole, imidazole, or pyridine, which rings are substituted with 1 or 2 groups R^{2a} or C₁-C₂-alkoxy-C₁-C₂-alkyl.

R¹ is preferably halomethyl, such as CF₃.

X is preferably N—R³.

R³ is preferably H, C₁-C₆-alkoxycarbonyl, or C₁-C₆-alkyl, which alkyl is unsubstituted or substituted with CN, C₃-C₆-cycloalkyl, and/or C₁-C₆-alkoxy. R³ being H, C₂H₅, or CH₂OCH₃ is particularly preferred.

In another embodiment R³ is OH, C₁-C₄-alkoxy, or C₁-C₄-alkoxycarbonyl.

In another embodiment X is O.

In one embodiment G is a group GA:

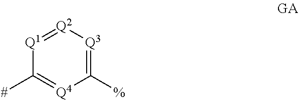

wherein
Q¹, Q², Q³, Q⁴ are N or CR²; provided that at least three are CR²;
R² are independently from each other preferably H, halogen, CN, C₁-C₄-alkyl, C₁-C₄-cycloalkyl, C₁-C₄-haloalkyl, C₁-C₄-halocycloalkyl, C₁-C₃-alkoxy, C₁-C₃-haloalkoxy, S(O)ₘ—C₁-C₃-alkyl, S(O)ₘ—C₁-C₃-haloalkyl.

G is preferably a group G1, G2, G3, or G4:

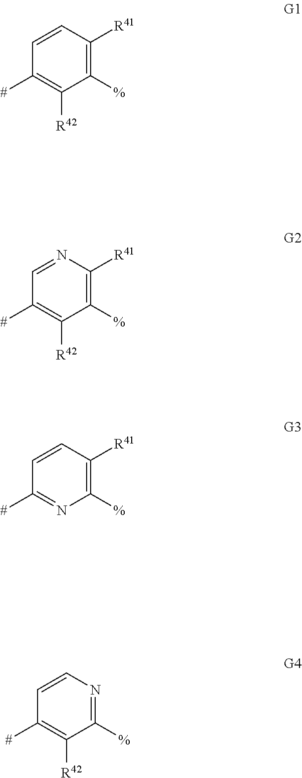

wherein # is the bond to X, % is the bond to Y which is a direct bond, and R⁴¹ and R⁴² are H or a group R², preferably H, or halogen, CN, halomethyl, halomethoxy. In a particularly preferred embodiment R⁴¹ is CN, and halogen such as Cl, and R⁴² is H.

In a preferred embodiment G is G1 and R⁴² is H.

In a preferred embodiment Q is a pyrazole, imidazole, or triazole; preferably selected from 1-pyrazole, 1-imidazole, and 1,2,4-triazole, which are unsubstituted or substituted with CN, C₁-C₄-alkyl, C₁-C₄-haloalkyl, C₁-C₄-haloalkoxy, S(O)ₘ—C₁-C₄-alkyl, S(O)ₘ—C₁-C₄-haloalkyl, NR¹⁵C(═O)R²⁶, or C(═O)NR^{25a}R^{25b}, wherein R^{25a} and R^{25b} are preferably H, C₁-C₄-alkyl, C₁-C₄-haloalkyl, and C₃-C₆-cycloalkyl.

A preferred embodiment are compounds of formula I, wherein
R¹ is C₁-C₂-haloalkyl;
W is phenyl, which partially or fully substituted with R²;
   R² is halogen, CN, OH, C₁-C₄-alkoxy, OR²¹, C₁-C₄-haloalkyl, C₁-C₃-alkoxy, C₁-C₃-haloalkoxy, C₁-C₃-alkyl-S(O)ₘ, C₁-C₃-haloalkyl-S(O)ₘ, C₁-C₃-alkoxy-C₁-C₄-alkyl;
X is NR³ or O;
R³ is H, C₁-C₄-alkyl, C₁-C₄-alkoxy-C₁-C₄-alkyl, C₁-C₄-alkoxycarbonyl, C₃-C₆-cycloalkyl-C₁-C₄-alkyl, C₂-C₄-alkenyl, C₂-C₄-alkynyl,
G is phenyl, which is unsubstituted, partially, or fully substituted with R²;
Q is pyrazole, imidazole, or triazole, Q being unsubstituted, or partially or fully substituted with CN, C₁-C₄-haloalkyl, C(═O)N(R^{12a})R^{12b}; wherein R^{12a}, R^{12b} are H, C₁-C₄-alkyl, C₁-C₄-haloalkyl.

A preferred embodiment are compounds of formula I which correspond to formula I′ as depicted below, wherein the variables are defined and preferred as above.

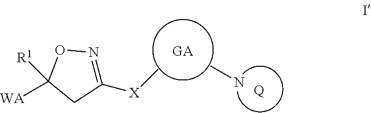

Another preferred embodiment are compounds of formula I which correspond to formula 1.1 as depicted below, wherein the variables are defined and preferred as above.

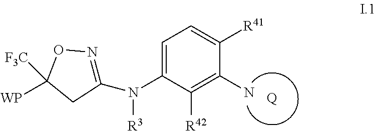

In particular with a view to their use, preference is given to the compounds of formula I compiled in the tables below, which compounds correspond to formula 1.1 wherein R⁴² denotes H. Each of the groups mentioned for a substituent in the tables is furthermore per se, independently of the combination in which it is mentioned, a particularly preferred aspect of the substituent in question.
Table 1: Compounds of formula 1.1 in which Q is 1-pyrazol-1-yl, and the other variables for a compound correspond in each case to one row of Table A
Table 2: Compounds of formula 1.1 in which Q is 4-CH₃-pyrazol-1-yl, and the other variables for a compound correspond in each case to one row of Table A
Table 3: Compounds of formula 1.1 in which Q is 4-CF₃-pyrazol-1-yl, and the other variables for a compound correspond in each case to one row of Table A
Table 4: Compounds of formula 1.1 in which Q is 4-CN-pyrazol-1-yl, and the other variables for a compound correspond in each case to one row of Table A
Table 5: Compounds of formula 1.1 in which Q is 4-CONHC₂H₅-pyrazol-1-yl, and the other variables for a compound correspond in each case to one row of Table A
Table 6: Compounds of formula 1.1 in which Q is 4-CONHCH₂CF₃-pyrazol-1-yl, and the other variables for a compound correspond in each case to one row of Table A
Table 7: Compounds of formula 1.1 in which Q is imidazol-1-yl, and the other variables for a compound correspond in each case to one row of Table A
Table 8: Compounds of formula 1.1 in which Q is 4-CH₃-imidazol-1-yl, and the other variables for a compound correspond in each case to one row of Table A
Table 9: Compounds of formula 1.1 in which Q is 4-CF₃-imidazol-1-yl, and the other variables for a compound correspond in each case to one row of Table A
Table 10: Compounds of formula 1.1 in which Q is 4-CN-imidazol-1-yl, and the other variables for a compound correspond in each case to one row of Table A
Table 11: Compounds of formula 1.1 in which Q is 4-CONHC₂H₅-imidazol-1-yl, and the other variables for a compound correspond in each case to one row of Table A
Table 12: Compounds of formula 1.1 in which Q is 4-CONHCH₂CF₃-imidazol-1-yl, and the other variables for a compound correspond in each case to one row of Table A
Table 13: Compounds of formula 1.1 in which Q is 1,2,4-triazolyl, and the other variables for a compound correspond in each case to one row of Table A

TABLE A

| No. | WP | R³ | R⁴¹ |
|---|---|---|---|
| A-1 | W-8 | H | Cl |
| A-2 | W-9 | H | Cl |
| A-3 | W-10 | H | Cl |
| A-4 | W-11 | H | Cl |
| A-5 | W-18 | H | Cl |
| A-6 | W-20 | H | Cl |
| A-7 | W-26 | H | Cl |
| A-8 | W-29 | H | Cl |
| A-9 | W-8 | CH₃ | Cl |
| A-10 | W-9 | CH₃ | Cl |
| A-11 | W-10 | CH₃ | Cl |
| A-12 | W-11 | CH₃ | Cl |
| A-13 | W-18 | CH₃ | Cl |
| A-14 | W-20 | CH₃ | Cl |
| A-15 | W-26 | CH₃ | Cl |
| A-16 | W-29 | CH₃ | Cl |
| A-17 | W-8 | CH₂CH₃ | Cl |
| A-18 | W-9 | CH₂CH₃ | Cl |
| A-19 | W-10 | CH₂CH₃ | Cl |
| A-20 | W-11 | CH₂CH₃ | Cl |
| A-21 | W-18 | CH₂CH₃ | Cl |
| A-22 | W-20 | CH₂CH₃ | Cl |
| A-23 | W-26 | CH₂CH₃ | Cl |
| A-24 | W-29 | CH₂CH₃ | Cl |
| A-25 | W-8 | CH₂OCH₃ | Cl |
| A-26 | W-9 | CH₂OCH₃ | Cl |
| A-27 | W-10 | CH₂OCH₃ | Cl |
| A-28 | W-11 | CH₂OCH₃ | Cl |
| A-29 | W-18 | CH₂OCH₃ | Cl |
| A-30 | W-20 | CH₂OCH₃ | Cl |
| A-31 | W-26 | CH₂OCH₃ | Cl |
| A-32 | W-29 | CH₂OCH₃ | Cl |
| A-33 | W-8 | H | CH₃ |
| A-34 | W-9 | H | CH₃ |
| A-35 | W-10 | H | CH₃ |
| A-36 | W-11 | H | CH₃ |
| A-37 | W-18 | H | CH₃ |
| A-38 | W-20 | H | CH₃ |
| A-39 | W-26 | H | CH₃ |
| A-40 | W-29 | H | CH₃ |
| A-41 | W-8 | CH₃ | CH₃ |
| A-42 | W-9 | CH₃ | CH₃ |
| A-43 | W-10 | CH₃ | CH₃ |
| A-44 | W-11 | CH₃ | CH₃ |
| A-45 | W-18 | CH₃ | CH₃ |
| A-46 | W-20 | CH₃ | CH₃ |
| A-47 | W-26 | CH₃ | CH₃ |
| A-48 | W-29 | CH₃ | CH₃ |
| A-49 | W-8 | CH₂CH₃ | CH₃ |
| A-50 | W-9 | CH₂CH₃ | CH₃ |
| A-51 | W-10 | CH₂CH₃ | CH₃ |
| A-52 | W-11 | CH₂CH₃ | CH₃ |
| A-53 | W-18 | CH₂CH₃ | CH₃ |
| A-54 | W-20 | CH₂CH₃ | CH₃ |
| A-55 | W-26 | CH₂CH₃ | CH₃ |
| A-56 | W-29 | CH₂CH₃ | CH₃ |
| A-57 | W-8 | CH₂OCH₃ | CH₃ |
| A-58 | W-9 | CH₂OCH₃ | CH₃ |
| A-59 | W-10 | CH₂OCH₃ | CH₃ |
| A-60 | W-11 | CH₂OCH₃ | CH₃ |
| A-61 | W-18 | CH₂OCH₃ | CH₃ |
| A-62 | W-20 | CH₂OCH₃ | CH₃ |
| A-63 | W-26 | CH₂OCH₃ | CH₃ |
| A-64 | W-29 | CH₂OCH₃ | CH₃ |
| A-65 | W-8 | H | OCF₃ |
| A-66 | W-9 | H | OCF₃ |
| A-67 | W-10 | H | OCF₃ |
| A-68 | W-11 | H | OCF₃ |
| A-69 | W-18 | H | OCF₃ |
| A-70 | W-20 | H | OCF₃ |
| A-71 | W-26 | H | OCF₃ |
| A-72 | W-29 | H | OCF₃ |
| A-73 | W-8 | CH₃ | OCF₃ |
| A-74 | W-9 | CH₃ | OCF₃ |
| A-75 | W-10 | CH₃ | OCF₃ |
| A-76 | W-11 | CH₃ | OCF₃ |
| A-77 | W-18 | CH₃ | OCF₃ |
| A-78 | W-20 | CH₃ | OCF₃ |
| A-79 | W-26 | CH₃ | OCF₃ |
| A-80 | W-29 | CH₃ | OCF₃ |
| A-81 | W-8 | CH₂CH₃ | OCF₃ |
| A-82 | W-9 | CH₂CH₃ | OCF₃ |
| A-83 | W-10 | CH₂CH₃ | OCF₃ |
| A-84 | W-11 | CH₂CH₃ | OCF₃ |
| A-85 | W-18 | CH₂CH₃ | OCF₃ |
| A-86 | W-20 | CH₂CH₃ | OCF₃ |
| A-87 | W-26 | CH₂CH₃ | OCF₃ |
| A-88 | W-29 | CH₂CH₃ | OCF₃ |
| A-89 | W-8 | CH₂OCH₃ | OCF₃ |
| A-90 | W-9 | CH₂OCH₃ | OCF₃ |
| A-91 | W-10 | CH₂OCH₃ | OCF₃ |
| A-92 | W-11 | CH₂OCH₃ | OCF₃ |
| A-93 | W-18 | CH₂OCH₃ | OCF₃ |
| A-94 | W-20 | CH₂OCH₃ | OCF₃ |
| A-95 | W-26 | CH₂OCH₃ | OCF₃ |
| A-96 | W-29 | CH₂OCH₃ | OCF₃ |
| A-97 | W-8 | H | CN |
| A-98 | W-9 | H | CN |
| A-99 | W-10 | H | CN |
| A-100 | W-11 | H | CN |
| A-101 | W-18 | H | CN |
| A-102 | W-20 | H | CN |
| A-103 | W-26 | H | CN |
| A-104 | W-29 | H | CN |
| A-105 | W-8 | CH₃ | CN |
| A-106 | W-9 | CH₃ | CN |
| A-107 | W-10 | CH₃ | CN |
| A-108 | W-11 | CH₃ | CN |
| A-109 | W-18 | CH₃ | CN |
| A-110 | W-20 | CH₃ | CN |
| A-111 | W-26 | CH₃ | CN |
| A-112 | W-29 | CH₃ | CN |
| A-113 | W-8 | CH₂CH₃ | CN |
| A-114 | W-9 | CH₂CH₃ | CN |
| A-115 | W-10 | CH₂CH₃ | CN |
| A-116 | W-11 | CH₂CH₃ | CN |
| A-117 | W-18 | CH₂CH₃ | CN |
| A-118 | W-20 | CH₂CH₃ | CN |
| A-119 | W-26 | CH₂CH₃ | CN |
| A-120 | W-29 | CH₂CH₃ | CN |
| A-121 | W-8 | CH₂OCH₃ | CN |
| A-122 | W-9 | CH₂OCH₃ | CN |
| A-123 | W-10 | CH₂OCH₃ | CN |
| A-124 | W-11 | CH₂OCH₃ | CN |
| A-125 | W-18 | CH₂OCH₃ | CN |
| A-126 | W-20 | CH₂OCH₃ | CN |
| A-127 | W-26 | CH₂OCH₃ | CN |
| A-128 | W-29 | CH₂OCH₃ | CN |

The term “compound(s) of the invention” refers to compound(s) of formula 1, or “compound(s) I”, and includes their salts, tautomers, stereoisomers, and N-oxides.

The invention also relates to agrochemical compositions comprising an auxiliary and at least one compound 1.

An agrochemical composition comprises a pesticidally effective amount of a compound 1.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-substituted fatty acid amides, amine oxides, esters, sugar-based surfactants, polymeric surfactants. Suitable cationic surfactants are quaternary surfactants.

The agrochemical compositions generally comprise between 0.01 and 95%, preferably between 0.1 and 90%, and most preferably between 0.5 and 75%, by weight of active substance.

The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grapefruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes, sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aerosol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plu-tella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Mosquitoes (Diptera), e.g. Aedes aegypti, A. albopictus, A. vexans, Anastrepha ludens, Anopheles maculipennis, A. crucians, A. albimanus, A. gambiae, A. freeborni, A. leucosphyrus, A. minimus, A. quadrimaculatus;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne sp., Pratylenchus spp., Caenorhabditis elegans.

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets, or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition, the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending, or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### A. PREPARATION EXAMPLES

With appropriate modification of the starting materials, the procedures given in the synthesis description were used to obtain further compounds I. The compounds obtained in this manner are listed in the table that follows, together with physical data.

The products shown below were characterized by melting point determination, by NMR spectroscopy or by the masses ([m/z]) or retention time (RT; [min.]) determined by HPLC-MS or HPLC spectrometry.

HPLC-MS=high performance liquid chromatography-coupled mass spectrometry;

HPLC method A: Shimadzu LC2010, Column: Waters XBridge C18,150 mm×4.6 mm ID×5μ; Mobile Phase: A: water+0.1% TFA; B: acetonitrile+0.1% TFA; Temperature: 40° C.; Gradient: 10% B to 100% B in 5 min; 100% B 2 min; 10% B 3 min; Flow: 1.4 ml/min; Run Time: 10 min; PDA detector.

### Example 1: Synthesis of N-[4-chloro-3-[4-(trifluoromethyl)pyrazol-1-yl]phenyl]-5-(3,5-dichloro-4-fluoro-phenyl)-N-(methoxymethyl)-5-(trifluoromethyl)-4H-isoxazol-3-amine [I-23]

Step 1: To a solution of 4-bromo-1-chloro-2-fluoro-benzene (2.00 g, 9.55 mmol) and 4-(trifluoromethyl)-1H-pyrazole (1.30 g, 9.55 mmol) in DMSO (50 mL) at ambient temperature (20-25° C.) was added Cs₂CO₃ (9.4 g, 28.65 mmol), and the resulting mixture was then stirred at 110° C. for 5 h. Then the mixture was poured into H₂O (100 mL), the aqueous phase was extracted with EtOAc (3×50 mL), and the combined organic extracts were washed with NaCl sat. aq. (2×50 mL), dried over Na₂SO₄, filtered, and concentrated under reduced pressure. Purification by column chromatography (petroleum ether/EtOAc=10:1 to 1:1) afforded 1-(5-bromo-2-chloro-phenyl)-4-(trifluoromethyl)pyrazole (1.60 g, 51%) as a white solid.

¹H-NMR (400 MHz, DMSO-d6, RT): δ 8.88 (s, 1H), 8.25 (s, 1H), 7.97 (d, J=2.4 Hz, 1H), 7.83-7.77 (m, 1H), 7.72-7.67 (m, 1H).

Step 2: To a solution of 1-(5-bromo-2-chloro-phenyl)-4-(trifluoromethyl)pyrazole (500 mg, 1.54 mmol) and 5-(3,5-dichloro-4-fluoro-phenyl)-5-(trifluoromethyl)-4H-isoxazol-3-amine (486 mg, 1.54 mmol, synthesized as described in PCT/EP2022/052181) in 1,4-dioxane (10 mL) at 20-25° C. under an atmosphere of N₂ was added Cs₂CO₃ (1.50 g, 4.62 mmol), Pd₂(dba)₃ (145 mg, 0.15 mmol, CAS-No. 51364-51-3), and Xantphos (166 mg, 0.30 mmol, CAS-No. 161265-03-8). The mixture was heated to reflux and stirred at that temperature for 16 h. Then the mixture was filtered and the filtrate was poured into H₂O (30 mL). The aqueous phase was extracted with EtOAc (3×10 mL), and the combined organic extracts were washed with NaCl solution (2×10 mL), dried over Na₂SO₄, filtered, and concentrated under reduced pressure. The residue was purified by Prep-HPLC to give N-[4-chloro-3-[4-(trifluoromethyl)pyrazol-1-yl]phenyl]-5-(3,5-dichloro-4-fluoro-phenyl)-5-(trifluoromethyl)-4H-isoxazol-3-amine [I-14](142 mg, 17%) as a yellow solid.

¹H-NMR (400 MHz, DMSO-d6, RT): δ 9.79 (s, 1H), 8.87 (s, 1H), 8.24 (s, 1H), 7.83 (d, J=6.3 Hz, 2H), 7.77 (d, J=2.6 Hz, 1H), 7.63 (d, J=8.8 Hz, 1H), 7.47 (dd, J=2.6, 8.9 Hz, 1H), 4.02-3.86 (m, 2H).

Step 3: To a solution of N-[4-chloro-3-[4-(trifluoromethyl)pyrazol-1-yl]phenyl]-5-(3,5-dichloro-4-fluoro-phenyl)-5-(trifluoromethyl)-4H-isoxazol-3-amine [I-14](100 mg, 0.178 mmol) in CH₂Cl₂ (5.0 mL) at 0° C. was added paraformaldehyde (57 mg, 1.1 mmol), and Me₃SiCl (109 mg, 1.1 mmol), before the cooling bath was removed and the mixture stirred at 20-25° C. for 16 h. Then the mixture was re-cooled to 0° C., MeOH (0.3 mL) was added, and stirring was continued for another 0.5 h. The reaction mixture was quenched with NaHCO₃ solution (sat. aqueous, 10 mL), the aqueous phase was extracted with EtOAc (3×10 mL), and the combined organic extracts were dried over Na₂SO₄, filtered, and concentrated under reduced pressure. Purification by column chromatography (SiO₂, EtOAc/cyclohexane 0:100 to 50:50, gradient) afforded the title N-[4-chloro-3-[4-(trifluoromethyl)pyrazol-1-yl]phenyl]-5-(3,5-dichloro-4-fluoro-phenyl)-N(methoxymethyl)-5-(trifluoromethyl)-4H-isoxazol-3-amine (30 mg, 28%).

¹H-NMR (400 MHz, CDCl₃, RT): δ 8.36 (d, J=0.6 Hz, 1H), 8.01 (d, J=0.6 Hz, 1H), 7.57 (d, J=8.7 Hz, 1H), 7.57 (d, J=2.6 Hz, 1H), 7.50 (d, J=6.0 Hz, 2H), 7.37 (dd, J=8.7, 2.6 Hz, 1H), 4.89 (s, 2H), 3.82 (d, J=16.7 Hz, 1H), 3.43 (d, J=16.4 Hz, 1H), 3.41 (s, 3H).

### Example 2: Synthesis of 1-[2-chloro-5-[[5-(3,5-dichloro-4-fluoro-phenyl)-5-(trifluoromethyl)-4Hisoxazol-3-yl]amino]phenyl]-N-(2,2,2-trifluoroethyl)pyrazole-4-carboxamide [I-18]

Step 1: To a solution of 4-bromo-1-chloro-2-fluoro-benzene (10.0 g, 47.9 mmol) and methyl 1Hpyrazole-4-carboxylate (9.04 g, 71.8 mmol) in DMSO (100 mL) at 20-25° C. was added Cs₂CO₃ (47.1 g, 144 mmol), and the resulting suspension was stirred at 120° C. for 16 h. The reaction mixture was quenched with H₂O (300 mL), the aqueous phase was extracted with EtOAc (2×100 mL), and the combined organic extracts were washed with NaCl solution (1×100 mL), dried over Na₂SO₄, filtered, and concentrated under reduced pressure. Purification by column chromatography (petroleum ether/EtOAc=10:1 to 1:1) afforded methyl 1-(5-bromo-2-chloro-phenyl)pyrazole-4-carboxylate (8.34 g, 55%) as a white solid.

¹H-NMR: (400 MHz, DMSO-d6, RT): δ 8.79 (s, 1H), 8.18 (s, 1H), 7.93 (d, J=2.3 Hz, 1H), 7.79-7.75 (m, 1H), 7.70-7.66 (m, 1H), 3.80 (s, 3H).

Step 2: To a solution of methyl 1-(5-bromo-2-chloro-phenyl)pyrazole-4-carboxylate (1.80 g, 5.70 mmol) and 5-(3,5-dichloro-4-fluoro-phenyl)-5-(trifluoromethyl)-4H-isoxazol-3-amine (486 mg, 1.54 mmol, synthesized as described in PCT/EP2022/052181) in 1,4-dioxane (20 mL) at 20-25° C. under an atmosphere of N₂ was added Cs₂CO₃ (5.57 g, 17.1 mmol), Xantphos (632 mg, 1.14 mmol, CAS-No. 161265-03-8) and Pd₂(dba)₃ (552 mg, 0.57 mmol, CAS-No. 51364-51-3), before the mixture was stirred at reflux temperature for 16 h. Then the reaction mixture was quenched with H₂O (60 mL), the aqueous phase was extracted with EtOAc (2×20 mL), and the combined organic extracts were washed with NaCl solution (sat. aqueous, 1×20 mL), dried over Na₂SO₄, filtered, and concentrated under reduced pressure. Purification by Prep-HPLC afforded methyl 1-[2-chloro-5-[[5-(3,5-dichloro-4-fluoro-phenyl)-5-(trifluoromethyl)-4H-isoxazol-3-yl]amino]phenyl]pyrazole-4-carboxylate (800 mg, 25%) as a yellow solid.

¹H-NMR (400 MHz, DMSO-d6, RT): δ 9.76 (s, 1H), 8.74 (s, 1H), 8.17 (s, 1H), 7.86-7.74 (m, 3H), 7.61 (d, J=8.8 Hz, 1H), 7.46 (dd, J=2.6, 8.9 Hz, 1H), 4.00-3.86 (m, 2H), 3.80 (s, 3H).

Step 3: To the solution of methyl 1-[2-chloro-5-[[5-(3,5-dichloro-4-fluoro-phenyl)-5-(trifluoromethyl)-4H-isoxazol-3-yl]amino]phenyl]pyrazole-4-carboxylate (800 mg, 1.45 mmol) in THF (4.0 mL) at 20-25° C. was added a solution of NaOH (114 mg, 2.90 mmol) in H₂O (4.0 mL), and the resulting mixture was then stirred at 40° C. for 16 h. The mixture was adjusted to pH=3 with HCl solution (2N in H₂O), the aqueous phase was extracted with EtOAc (3×20 mL), and the combined organic extracts were dried over Na₂SO₄, filtered, and concentrated under reduced pressure to give 1-[2-chloro-5-[[5-(3,5-dichloro-4-fluoro-phenyl)-5-(trifluoromethyl)-4H-isoxazol-3-yl]amino]phenyl]pyrazole-4-carboxylic acid (800 mg, crude) as a yellow solid.

¹H-NMR (400 MHz, DMSO-d6, RT): δ 12.7 (br s, 1H), 9.75 (s, 1H), 8.63 (s, 1H), 8.09 (s, 1H), 7.83 (d, J=6.3 Hz, 2H), 7.76 (d, J=2.6 Hz, 1H), 7.61 (d, J=8.9 Hz, 1H), 7.45 (dd, J=2.6, 8.9 Hz, 1H), 4.00-3.86 (m, 2H).

Step 4: To a solution of 1-[2-chloro-5-[[5-(3,5-dichloro-4-fluoro-phenyl)-5-(trifluoromethyl)-4Hisoxazol-3-yl]amino]phenyl]pyrazole-4-carboxylic acid (800 mg, 1.49 mmol) in CH₂Cl₂ (10 mL) was added 2,4,6-Tripropyl-1,3,5,2λ5,4λ5,6λ5-trioxatriphosphinane-2,4,6-trione (710 mg, 2.23 mmol, “T3P”, CAS-No. 68957-94-8) and Et₃N (452 mg, 4.47 mmol), before 2,2,2-trifluoroethanamine was added dropwise at 0° C. The mixture was stirred at 20-25° C. for 16 h. The reaction mixture was quenched with H₂O (40 mL), the aqueous phase was extracted with EtOAc (2×20 mL), and the combined organic extracts were washed with NaCl solution (sat. aqueous, 1×20 mL), dried over Na₂SO₄, filtered, and concentrated under reduced pressure. Purification by Prep-HPLC afforded the title 1-[2-chloro-5-[[5-(3,5-dichloro-4-fluoro-phenyl)-5-(trifluoromethyl)-4H-isoxazol-3-yl]amino]phenyl]-N-(2,2,2-trifluoroethyl)pyrazole-4-carboxamide [I-18](287 mg, 31%) as a white solid.

¹H-NMR (400 MHz, DMSO-d6, RT): δ 9.74 (s, 1H), 8.89 (br t, J=6.3 Hz, 1H), 8.69 (s, 1H), 8.22 (s, 1H), 7.87-7.76 (m, 3H), 7.61 (d, J=8.9 Hz, 1H), 7.44 (dd, J=2.6, 8.9 Hz, 1H), 4.13-4.04 (m, 2H), 4.00-3.86 (m, 2H).

### Example 3: Synthesis of 4-[[5-(3,5-dichloro-4-fluoro-phenyl)-5-(trifluoromethyl)-4H-isoxazol-3-yl]-ethyl-amino]-2-(1,2,4-triazol-1-yl)benzonitrile [I-20]

Step 1: To a solution of 4-[[5-(3,5-dichloro-4-fluoro-phenyl)-5-(trifluoromethyl)-4H-isoxazol-3-yl]amino]-2-(1,2,4-triazol-1-yl)benzonitrile [I-4](80.0 mg, 0.165 mmol, synthesized analogously as described above) and iodoethane (20 μL, 0.25 mmol) in MeCN (1.5 mL) at 20-25° C. was added K₂CO₃ (92.0 mg, 0.330 mmol), and the resulting reaction mixture was stirred at that temperature for 1d before it was heated at 50° C. over night. Then the reaction mixture was diluted with H₂O (20 mL), the aqueous phase was extracted with CH₂Cl₂ (3×20 mL), and the combined organic extracts were dried over Na₂SO₄, filtered, and concentrated under reduced pressure. Purification by column chromatography (SiO₂, EtOAc/cyclohexane 0:100 to 50:50, gradient) afforded the title 4-[[5-(3,5-dichloro-4-fluoro-phenyl)-5-(trifluoromethyl)-4H-isoxazol-3-yl]-ethyl-amino]-2-(1,2,4-triazol-1-yl)benzonitrile [I-20](40 mg, 47%).

¹H-NMR (400 MHz, CDCl₃, RT): δ 8.90 (s, 1H), 8.18 (s, 1H), 7.82 (d, J=8.5 Hz, 1H), 7.60 (d, J=2.3 Hz, 1H), 7.51 (d, J=6.0 Hz, 2H), 7.29 (dd, J=8.5, 2.2 Hz, 1H), 3.87 (app. qd, J=7.2, 3.5 Hz, 2H), 3.79 (d, J=16.6 Hz, 1H), 3.40 (d, J=16.6 Hz, 1H), 1.29 (t, J=7.1 Hz, 3H).

### Example 4: Synthesis of ethyl N-[4-chloro-3-[4-(trifluoromethyl)imidazol-1-yl]phenyl]-N-[5-(3,5-dichloro-4-fluoro-phenyl)-5-(trifluoromethyl)-4H-isoxazol-3-yl]carbamate [I-26]

Step 1: To a solution of N-[4-chloro-3-[4-(trifluoromethyl)imidazol-1-yl]phenyl]-5-(3,5-dichloro4-fluoro-phenyl)-5-(trifluoromethyl)-4H-isoxazol-3-amine [I-16](63.0 mg, 0.112 mmol, synthesized analogously as described above) in THF (2.5 mL) at 0° C. was added t-BuOK (13 mg, 0.12 mmol), and the resulting mixture was stirred at that temperature for 30 min. Then ethyl chloroformate (13 μL, 0.13 mmol) was added and the reaction mixture allowed to slowly reach 20-25° C. over 3 h. Then the reaction mixture was diluted with NaCl sat. aq. (10 mL), the aqueous phase was extracted with CH₂Cl₂ (3×10 mL), and the combined organic extracts were dried over Na₂SO₄, filtered, and concentrated under reduced pressure. Purification by column chromatography (SiO₂, EtOAc/cyclohexane 0:100 to 50:50, gradient) afforded the title 4-[[5-(3,5-dichloro-4-fluoro-phenyl)-5-(trifluoromethyl)-4H-isoxazol-3-yl]-ethyl-amino]-2-(1,2,4-triazol-1-yl)benzonitrile [I-26](39 mg, 55%).

¹H-NMR 400 MHz, CDCl₃, RT): δ 7.75 (s, 1H), 7.66 (d, J=8.6 Hz, 1H), 7.52 (s, 1H), 7.50 (d, J=6.0 Hz, 2H), 7.32 (dd, J=8.6, 2.5 Hz, 1H), 7.28 (d, J=2.4 Hz, 1H), 4.41 (d, J=18.3 Hz, 1H), 4.25 (q, J=7.1 Hz, 2H), 4.04 (d, J=18.3 Hz, 1H), 1.24 (t, J=7.1 Hz, 3H).

### Example 5: Synthesis of 3-[4-chloro-3-(1,2,4-triazol-1-yl)phenoxy]-5-(3,5-dichloro-4-fluorophenyl)-5-(trifluoromethyl)-4H-isoxazole [I-27]

Step 1: To a solution of 3-bromo-4-chloro-phenol (10.0 g, 48.1 mmol) in DMSO (100 mL) at 20-25° C. and under an atmosphere of N₂ was added 4H-1,2,4-triazole (10.0 g, 145 mmol), CuO (3.80 g, 48.2 mmol) and K₂CO₃ (33.3 g, 241 mmol). Then, the reaction mixture was heated to 145° C. and stirred at that temperature for 16 h. The mixture was filtered, and the filter cake was washed with EtOAc (3×50 mL). The filtrate was mixed with H₂O (200 mL), the aqueous phase was extracted with EtOAc (3×100 mL), and the combined organic extracts were washed with NaCl solution (3×50 mL), dried over Na₂SO₄, filtered, and concentrated under reduced pressure. Purification by column chromatography (petroleum ether/EtOAc=100:0 to 50:50, gradient) afforded 4-chloro-3-(1,2,4-triazol-1-yl)phenol (1.01 g, 11%) as a yellow solid.

¹H NMR (400 MHz, MeOH-d4, RT): δ 8.82 (s, 1H) 8.19 (s, 1H) 7.43 (d, J=8.8 Hz, 1H) 7.01 (d, J=2.9 Hz, 1H) 6.95 (dd, J=8.8, 2.9 Hz, 1H).

Step 2: To a solution of 4-chloro-3-(1,2,4-triazol-1-yl)phenol (1.01 g, 5.11 mmol) in NMP (80 mL) at 20-25° C. was added NaOH (204 mg, 5.11 mmol), and the reaction mixture was stirred at 90° C. for 1 h. Then, a solution of 3-bromo-5-(3,5-dichloro-4-fluoro-phenyl)-5-(trifluoromethyl)-4H-isoxazole (974 mg, 2.56 mmol, synthesized as described in PCT/EP2022/052181) in NMP (20 mL) was added above the mixture, and stirring was continued at 90° C. for 16 h. After that time, the reaction mixture was poured into H₂O (30 mL), the aqueous phase was extracted with EtOAc (3×50 mL), and the combined organic extracts were washed with NaCl solution (sat. aqueous, 1×20 mL), dried over Na₂SO₄, filtered, and concentrated under reduced pressure. Purification by column chromatography (petroleum ether/EtOAc=100:0 to 50:50, gradient) afforded the title 3-[4-chloro-3-(1,2,4-triazol-1-yl)phenoxy]-5-(3,5-dichloro-4-fluoro-phenyl)-5-(trifluoromethyl)-4H-isoxazole [I-27](55 mg, 2%).

¹H NMR (400 MHz, MeOH-d4, RT): δ 8.93 (s, 1H) 7.69-7.78 (m, 4H) 8.24 (s, 1H) 7.56 (dd, J=8.9, 2.8 Hz, 1H) 4.09 (d, J=17.8 Hz, 1H) 3.91 (d, J=17.8 Hz, 1H).

TABLE I.1
compounds of formula I (R¹ = CF₃)

| | | | | | | phys. data | |
|---|---|---|---|---|---|---|---|
| | | | | | | [HPLC Rt [min], | HPLC |
| No. | WP | X | R³ | G | Q | M + H [m/z]] | method |
| I-1 | W-9 | NR³ | H | G1, R⁴¹: CN R⁴²: H | 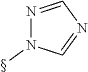 | 1.250, 466.7 | A |
| I-2 | W-9 | NR³ | H | 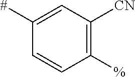 | 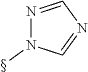 | 1.252, 466.7 | A |
| I-3 | W-9 | NR³ | H | 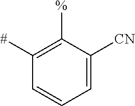 | 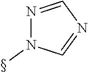 | 1.282, 466.9 | A |
| I-4 | W-11 | NR³ | H | G1, R⁴¹: CN R⁴²: H | 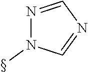 | 1.289, 484.8 | A |
| I-5 | W-18 | NR³ | H | G1, R⁴¹: CN R⁴²: H | 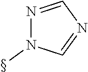 | 1.184, 467.0 | A |
| I-6 | W-8 | NR³ | H | G1, R⁴¹: CN R⁴²: H | 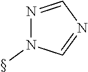 | 1.182, 450.9 | A |
| I-7 | W-9 | NR³ | CH₃ | G1, R⁴¹: CN R⁴²: H | 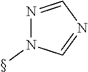 | 1.260, 481.0 | A |
| I-8 | W-26 | NR³ | H | G1, R⁴¹: CN R⁴²: H | 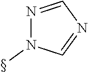 | 1.205, 485.0 | A |
| I-9 | W-8 | NR³ | CH₃ | G1, R⁴¹: CN R⁴²: H | 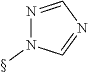 | 1.191, 465.2 | A |
| I-10 | W-8 | NR³ | C₂H₅ | G1, R⁴¹: CN R⁴²: H | 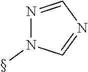 | 1.230, 479.2 | A |
| I-11 | W-8 | NR³ | CH₂—c—C₃H₅ | G1, R⁴¹: CN R⁴²: H | 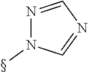 | 1.282, 505.3 | A |
| I-12 | W-8 | NR³ | CH₂—C≡CH | G1, R⁴¹: CN R⁴²: H | 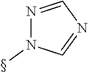 | 1.207, 489.2 | A |
| I-13 | W-11 | NR³ | H | G1, R⁴¹: Cl R⁴²: H | 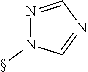 | 1.307, 495.8 | A |
| I-14 | W-11 | NR³ | H | G1, R⁴¹: Cl R⁴²: H | 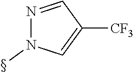 | 1.496, 562.8 | A |
| I-15 | W-11 | NR³ | H | G1, R⁴¹: Cl R⁴²: H | 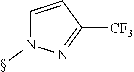 | 1.509, 562.9 | A |
| I-16 | W-11 | NR³ | H | G1, R⁴¹: Cl R⁴²: H | 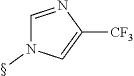 | 1.444, 562.9 | A |
| I-17 | W-11 | NR³ | H | G1, R⁴¹: Cl R⁴²: H | 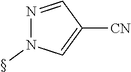 | 1.387, 518.0 | A |
| I-18 | W-11 | NR³ | H | G1, R⁴¹: Cl R⁴²: H | 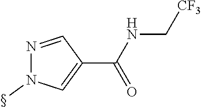 | 1.324, 618.3 | A |
| I-19 | W-11 | NR³ | C₂H₅ | G1, R⁴¹: Cl R⁴²: H | 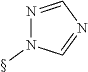 | 1.392, 523.8 | A |
| I-20 | W-11 | NR³ | C₂H₅ | G1, R⁴¹: CN R⁴²: H | 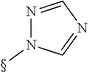 | 1.363, 512.8 | A |
| I-21 | W-11 | NR³ | CH₂OCH₃ | G1, R⁴¹: Cl R⁴²: H | 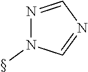 | 1.340, 538.3 | A |
| I-22 | W-11 | NR³ | CH₂OCH₃ | G1, R⁴¹: CN R⁴²: H | 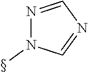 | 1.323, 528.9 | A |
| I-23 | W-11 | NR³ | CH₂OCH₃ | G1, R⁴¹: Cl R⁴²: H | 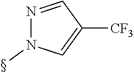 | 1.511, 601.9 | A |
| I-24 | W-11 | NR³ | CH₂OCH₃ | G1, R⁴¹: Cl R⁴²: H | 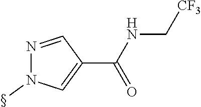 | 1.407, 672.9 | A |
| I-25 | W-11 | NR³ | C(═O)OC₂H₅ | G1, R⁴¹: Cl R⁴²: H | 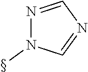 | 1.382, 567.9 | A |
| I-26 | W-11 | NR³ | C(═O)OC₂H₅ | G1, R⁴¹: Cl R⁴²: H | 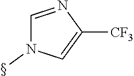 | 1.484, 634.8 | A |
| I-27 | W-11 | O | — | G1, R⁴¹: Cl R⁴²: H | 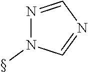 | 1.345, 496.6 | A |
| I-28 | W-11 | NR³ | H | G1, R⁴¹: OCF₃ R⁴²: H | 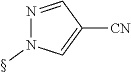 | 1.442, 567.8 | A |
| I-29 | W-11 | NR³ | H | G1, R⁴¹: Cl R⁴²: H | 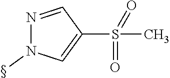 | 1.326, 572.6 | A |
| I-30 | W-11 | NR³ | H | G1, R⁴¹: CN R⁴²: H | 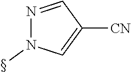 | 1.345, 508.8 | A |
| I-31 | W-11 | NR³ | C₂H₅ | G1, R⁴¹: Cl R⁴²: H | 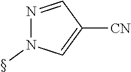 | 1.428, 548.2 | A |

§ = bond to the remainder of the molecule;
# = bond to X;
% = bond to Q

### BIOLOGICAL EXAMPLES

If not otherwise specified, the test solutions were prepared as follows:

The active compound was dissolved at the desired concentration in a mixture of 1:1 (vol:vol) distilled water:acetone. The test solution was prepared at the day of use.

Test solutions were prepared in general at a concentration of 2500 ppm (wt/vol).

B.1. Boll Weevil (Anthonomus grandis)

For evaluating control of boll weevil, the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom-built micro atomizer, at two replications.

After application, microtiter plates were incubated at about 25±1° C. and about 75±5% relative humidity for 5 days. Egg and larval mortality were then visually assessed.

In this test, compounds, I-1, I-2, I-4, I-5, I-6, I-7, I-9, I-10, I-11, I-12, I-13, I-14, I-16, I-17, I-18, I-19, I-20, I-21, I-26, I-27, I-28, I-29, I-30, and I-31 resp., at 2500 ppm showed at least 75% mortality in comparison with untreated controls.

B.2. Green Peach Aphid (Myzus persicae) (Mixed Life Stages)

For evaluating control of Green Peach Aphid through systemic means the test unit consisted of 96-well-microtiter plates containing liquid artificial diet under an artificial membrane. The compounds were formulated using a solution containing 75% v/v water and 25% v/v DMSO. Different concentrations of formulated compounds were pipetted into the aphid diet, using a custom-built pipette, at two replications. After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, I-1, I-4, I-5, I-6, I-7, I-9, I-10, I-11, I-12, I-13, I-14, I-16, I-17, I-18, I-19, and I-21 I-26, I-27, I-28, I-29, and I-30 resp., at 2500 ppm showed at least 75% mortality in comparison with untreated controls.

B.3. Tobacco Budworm (Heliothis virescens)

For evaluating control of Tobacco Budworm, the test unit consisted of 96-well-microtiter plates containing an insect diet and 15-25 H. virescens eggs. The compounds were formulated using a solution containing 75% v/v water and 25% v/v DMSO. Different concentrations of formulated compounds were sprayed onto the insect diet at 10 μl, using a custom-built micro atomizer, at two replications. After application, microtiter plates were incubated at about 28±1° C. and about 80±5% relative humidity for 5 days. Egg and larval mortality was then visually assessed.

In this test, compounds I-1, I-2, I-4, I-5, I-6, I-7, I-9, I-10, I-11, I-12, I-13, I-14, I-15, I-16, I-17, I-18, I-19, I-20, I-21, I-25, I-26, I-27, I-28, I-29, I-30, and I-31, resp., at 2500 ppm showed at least 75% mortality in comparison with untreated controls.

B.4. Yellow Fever Mosquito (Aedes aegypti)

For evaluating control of Yellow Fever Mosquito, the test unit consisted of 96-well-microtiter plates containing 200 μl of tap water per well and 5-15 freshly hatched A. aegypti larvae. The active compounds were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

After application, microtiter plates were incubated at 28±1° C., 80±5% RH for 2 days. Larval mortality was then visually assessed.

In this test, compounds I-1, I-2, I-4, I-5, I-6, I-7, I-9, I-10, I-11, I-12, I-13, I-14, I-15, I-16, I-17, I-19, I-20, I-21, I-25, I-26, I-27, I-28, I-29, I-30, and I-31, resp., at 2500 ppm showed at least 75% mortality in comparison with untreated controls.

B.5. Diamond Back Moth (Plutella xylostella)

The active compound was dissolved at the desired concentration in a mixture of 1:1 (vol:vol) distilled water:acetone. Surfactant (Kinetic) was added at a rate of 0.01% (vol/vol). The test solution was prepared at the day of use. Cabbage leaf discs (60 mm in diameter) were dipped in test solution and air-dried. Treated leaves were placed in petri dishes lined with moistened filter paper and inoculated with ten 3rd instar larvae. Mortality was recorded 72 hours after treatment. Feeding damages were also recorded using a scale of 0-100%.

In this test, compounds I-1, I-2, I-4, I-5, I-6, I-7, I-8, I-9, I-10, I-11, I-12, I-13, I-14, I-16, I-17, I-18, I-19, I-20, I-21, I-22, I-23, I-24, I-25, and I-26, resp., at 300 ppm showed at least 75% mortality in comparison with untreated controls.

B.6. Orchid Thrips (Dichromothrips corbetti)

Dichromothrips corbetti adults used for bioassay were obtained from a colony maintained continuously under laboratory conditions. For testing purposes, the test compound was diluted in a 1:1 mixture of acetone:water (vol:vol), plus Kinetic at a rate of 0.01% v/v.

Thrips potency of each compound was evaluated by using a floral-immersion technique. Each orchid petal was dipped into treatment solution and allowed to dry in Petri dishes. Treated petals were placed into individual re-sealable plastic along with about 20 adult thrips. All test arenas were held under dark condition and a temperature of about 28° C. for duration of the assay. The percent mortality was recorded 72 hours after treatment.

In this test, compounds I-1, I-4, I-5, I-6, I-7, I-8, I-9, I-10, I-11, I-12, I-14, I-15, I-16, I-17, I-18, I-19, I-20, I-21, I-22, I-23, I-24, I-25, I-26, I-27, I-29, I-30, and I-31, resp., at 300 ppm showed at least 75% mortality in comparison with untreated controls.

B.7. Southern Armyworm (Spodoptera eridania), 2nd Instar Larvae

The active compounds were formulated by a Tecan liquid handler in 100% cyclohexanone as a 10′000 ppm solution supplied in tubes. The 10′000 ppm solution was serially diluted in 100% cyclohexanone to make interim solutions. These served as stock solutions for which final dilutions were made by the Tecan in 50% acetone:50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photo-period) to prevent trapping of heat inside the bags. Mortality and reduced feeding were assessed 4 days after treatment, compared to untreated control plants.

In this test, compounds I-1, I-4, I-5, I-6, I-7, I-8, I-9, I-10, I-11, I-12, I-13, I-14, I-15, I-19, I-20, I-21, I-23, I-25, I-26, I-27, I-29, and I-30, resp., at 300 ppm showed at least 75% mortality in comparison with untreated controls.

B.8. Silverleaf Whitefly (Bemisia argentifoli) (Adults)

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 5 or 10 ml glass vials. A nonionic surfactant (Kinetic®) was included in the solution at a volume of 0.01% (v/v). The vials were then inserted into an automated electrostatic sprayer equipped with an atomizing nozzle for application to plants/insects. Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (14:10 light:dark photoperiod) to prevent trapping of heat inside the cup. Mortality was assessed 3 days after treatment, compared to untreated control 5 plants.

In this test, compounds I-8, I-13, I-14, I-15, I-21, and I-25, resp., at 300 ppm showed at least 75% mortality in comparison with untreated controls.

The beneficial activity of the compounds according to the invention over structurally close compounds known from prior art was demonstrated by the following comparative experiments:

| Structure | Example | B.5, 10 ppm | B.6, 10 ppm |
|---|---|---|---|
| 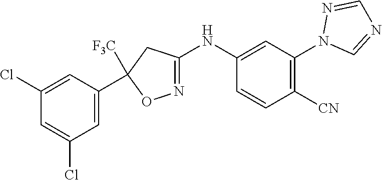 [SMILES: N#Cc1ccc(NC2=NOC(c3cc(Cl)cc(Cl)c3)(C(F)(F)F)C2)cc1-n1cncn1] | I-1 | 100 | 100 |
| 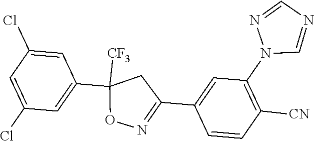 [SMILES: N#Cc1ccc(C2=NOC(c3cc(Cl)cc(Cl)c3)(C(F)(F)F)C2)cc1-n1cncn1] | WO2011/073444 Table 3937, No. D-539 | 0 | 0 |

| Structure | Example | B.5, 10 ppm | B.6, 10 ppm |
|---|---|---|---|
| 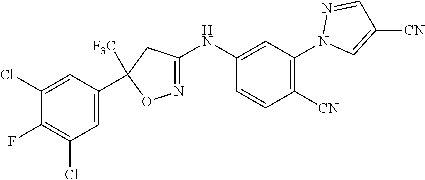 [SMILES: N#Cc1cnn(-c2cc(NC3=NOC(c4cc(Cl)c(F)c(Cl)c4)(C(F)(F)F)C3)ccc2C#N)c1] | I-17 | 100 | 97.5 |
| 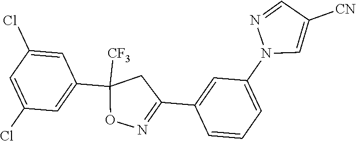 [SMILES: N#Cc1cnn(-c2cccc(C3=NOC(c4cc(Cl)cc(Cl)c4)(C(F)(F)F)C3)c2)c1] | WO2011/073444 Table C.1, No. I-4 | 0 | 0 |

1. A compound of formula I 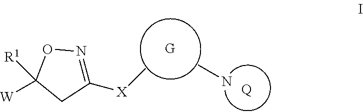
wherein
R¹ is C₁-C₂-haloalkyl;
W is phenyl, or pyridyl; wherein W is unsubstituted, partially or fully substituted with R²;
R² is halogen, OR²¹, NR²²R²³, CN, NO₂, Si(CH₃)₃, SbF₅, C₁-C₄-alkyl, C₁-C₄-haloalkyl, C₃-C₆-cycloalkyl, C₃-C₆-halocycloalkyl, C₂-C₄-alkenyl, C₂-C₄-haloalkenyl, C₂-C₄-alkynyl, C₂-C₄-haloalkynyl, C₁-C₃-alkoxy, C₁-C₃-haloalkoxy, C₁-C₃-alkyl-S(O)ₘ, C₁-C₃-haloalkyl-S(O)ₘ, C₃-C₆-cycloalkyl-S(O)ₘ, C₁-C₃-alkoxy-C₁-C₄-alkyl, C₁-C₃-haloalkoxy-C₁-C₄-alkyl, C₁-C₃-alkyl-S(O)ₘ—C₁-C₄-alkyl, C₁-C₃-haloalkyl-S(O)ₘ—C₁-C₄-alkyl; which groups are unsubstituted, partially or fully substituted with R²¹¹;
R²¹ H, C₁-C₆-alkyl, C₂-C₆-alkenyl, C₂-C₆-alkynyl, C₃-C₈-cycloalkyl, Si(C₁-C₄-alkyl)₃, C₁-C₃-alkyl-S(O)ₘ, C₃-C₆-cycloalkyl-S(O)ₘ, S(O)ₘR²⁴, which groups are unsubstituted, partially or fully substituted with R²¹¹;
R²², R²³ H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, which are unsubstituted or partially or fully substituted with R²²¹; or
C₁-C₆-alkyl-C(═O)OR²⁴, C₁-C₆-alkyl-C(=U)N(R^{25a})R²⁵¹, C₁-C₆-alkyl-C(═NR²⁵)N(R^{25a})R^{25b},
S(O)ₘR²⁴, S(O)ₘN(R^{25a})R^{25b}, C(=U)R²⁶, C(═O)OR²⁴, C(=U)N(R^{25a})R^{25b}, C(═S)SR²⁴, C(═NR²⁵)R²⁶;
C₃-C₈-cycloalkyl, C₃-C₈-halocycloalkyl;
R²² and R²³ form together with the nitrogen atom they are bonded to a 3-, 4-, 5-, or 6-membered fully unsaturated heterocycle, which heterocycle may additionally contain one heteroatom selected from N, O, and S(O)ₘ as ring members, and which heterocycle is unsubstituted or partially or fully substituted with R²²²; or
R²² and R²³ together form a group ═C(R²¹)₂, ═S(O)ₘ(R²⁴)₂, ═S(O)ₘR²⁴N(R^{25a})R^{25b};
U is O or S;
R²⁴ is H, Si(C₁-C₄-alkyl)₃, C₁-C₆-alkyl, C₂-C₆-alkenyl, C₂-C₆-alkynyl, which are unsubstituted or partially or fully halogenated and/or substituted with C₃-C₄-cycloalkyl, C₁-C₄-alkoxy, C₁-C₄-haloalkoxy, S(O)ₘ—C₁-C₄-alkyl, and oxo;
C₃-C₆-cycloalkyl which is unsubstituted or partially or fully halogenated and/or substituted with C₁-C₄-alkyl, C₃-C₄-cycloalkyl, C₁-C₄-alkoxy, C₁-C₄-haloalkoxy, S(O)ₘ—C₁-C₄-alkyl, and oxo;
phenyl, benzyl, pyridyl and phenoxy, which are unsubstituted or partially or fully halogenated and/or substituted with C₁-C₆-alkyl, C₁-C₆-haloalkyl, C₁-C₆-alkoxy, C₁-C₆-haloalkoxy, C₁-C₆-alkylthio, C₁-C₆-haloalkylthio, and (C₁-C₆-alkoxy)carbonyl;
R²⁵ is H, C₁-C₆-alkoxy, C₁-C₆-haloalkoxy, C(=U)R²⁶, C(═O)OR²⁴, C(═O)NH(C₁-C₄-alkyl), C(═O)N(C₁-C₄-alkyl)₂, S(O)ₘR²⁴, S(O)ₘ—C₁-C₄-alkyl, S(O)ₘ—C₁-C₄-haloalkyl, S(O)ₘ—C₃-C₆-cycloalkyl, Si(C₁-C₄-alkyl)₃, C₁-C₆-alkyl, C₃-C₆-alkenyl, C₃-C₆-alkynyl, which are unsubstituted or partially or fully halogenated and/or substituted with CN, C₁-C₄-alkoxy, C₁-C₄-haloalkoxy, S(O)ₘ—C₁-C₄-alkyl, S(O)ₘ—C₁-C₄-haloalkyl, C₃-C₆-cycloalkyl which is unsubstituted or substituted with 1 or 2 halogen and/or CN; phenyl, or 5-, 6-membered heteroaryl containing 1, 2, or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio; and oxo;
C₃-C₆-cycloalkyl which is unsubstituted or partially or fully halogenated and/or substituted with CN, C₁-C₄-alkyl, C₁-C₄-alkoxy, C₁-C₄-haloalkyl, C₁-C₄-haloalkoxy, S(O)ₘ—C₁-C₄-alkyl, S(O)ₘ—C₁-C₄-haloalkyl, C(═O)NH(C₁-C₄-alkyl), C(═O)N(C₁-C₄-alkyl)₂, phenyl, or 5-, 6-membered heteroaryl containing 1, 2, or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio;
phenyl, benzyl, pyridyl, phenoxy, which are unsubstituted or partially or fully halogenated and/or substituted with halogen, CN, NO₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, and (C₁-C₆-alkoxy)carbonyl; and a 3-, 4-, 5-, or 6-membered saturated, partially or fully unsaturated heterocycle comprising 1, 2, or 3 heteroatoms selected from N, O, and S(O)ₘ as ring members, where the heterocycle is optionally substituted with one or more R²²²;
R^{25a} and R^{25b} have the meanings given for R²⁵; or
R^{25a} and R^{25b} present on the same nitrogen atom may together form ═C(R²⁶)₂, ═S(O)ₘ(R²⁴)₂, or ═S(O)ₘR²⁴N(R^{25a})R^{25b}; or
R^{25a} and R^{25b}, together with the nitrogen atom to which they are bound, form a 3-, 4-, 5-, 6-, or 7-membered saturated, partially unsaturated or maximally unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo; or
R^{25a} and R²⁵, together with the nitrogen atoms to which they are bound in the group C(═NR²⁵)N(R^{25a})R^{25b}, form a 3-, 4-, 5-, 6-, or 7-membered saturated, partially unsaturated or maximally unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo;
R²⁶ is H, OH, SH, and C₁-C₆-alkyl, C₂-C₆-alkenyl, C₂-C₆-alkynyl, C₃-C₃-cycloalkyl, which are unsubstituted, partially or fully halogenated and/or substituted with 1, 2, or 3 groups CN, C₁-C₄-alkyl, C₃-C₄-cycloalkyl, C₁-C₄-haloalkyl, C₁-C₄-alkoxy, C₁-C₄-haloalkoxy, S(O)ₘ—C₁-C₄-alkyl, S(O)ₘ—C₁-C₄-haloalkyl, phenyl, or 3-, 4-, 5-, 6-, or 7-membered saturated, partially or fully unsaturated heterocycle containing 1, 2, or 3 heteroatoms N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio; and oxo;
C₁-C₆-alkoxy, C₁-C₆-haloalkoxy, S(O)ₘ—C₁-C₆-alkyl, S(O)ₘ—C₁-C₆-haloalkyl, Si(C₁-C₄-alkyl)₃, C(═O)N(R^{25a})R^{25b},
phenyl, benzyl, phenoxy, a 3-, 4-, 5-, 6-, or 7-membered saturated, partially or fully unsaturated heterocycle containing 1, 2, or 3 heteroatoms selected from N, O, and S(O)ₘ as ring members, which rings are unsubstituted or partially or fully halogenated and/or substituted with R²²²;
R²¹¹ halogen, CN, C₁-C₄-alkoxy, C₁-C₄-haloalkoxy, C₃-C₄-alkenyloxy, C₃-C₄-haloalkenyloxy, C₃-C₄-alkynyloxy, C₃-C₄-haloalkynyloxy, C₁-C₄-alkyl-S(O)ₘ, C₁-C₄-haloalkyl-S(O)ₘ, C₃-C₄-alkenyl-S(O)ₘ, C₃-C₄-haloalkenyl-S(O)ₘ, C₃-C₄-alkynyl-S(O)ₘ, C₃-C₄-haloalkynyl-S(O)ₘ, and oxo;
C₃-C₈-cycloalkyl, C₃—C-halocycloalkyl, C₃—C-cycloalkenyl, C₃-C₃-halocycloalkenyl;
R²²¹ CN, NO₂, OH, SH, SCN, SF₅, Si(C₁-C₄-alkyl)₃, C₁-C₆-alkoxy, C₁-C₆-haloalkoxy, C₁-C₆-alkyl-S(O)ₘ, C₁-C₆-haloalkyl-S(O)ₘ, C(═O)N(R^{25a})R^{25b};
C₃-C₈-cycloalkyl which is unsubstituted, partially or fully halogenated and/or partially or fully substituted with C₁-C₄-alkyl, C₃-C₄-cycloalkyl, C₁-C₄-alkoxy, C₁-C₄-haloalkoxy, and oxo; or
two R²²¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
R²²² is halogen, NO₂, CN, OH, SH, C₁-C₆-alkoxy, C₁-C₆-haloalkoxy, S(O)ₘC₁-C₄-alkyl, S(O)ₘ—C₁-C₄-haloalkyl, C₁-C₄-alkylcarbonyl, C₁-C₄-haloalkylcarbonyl, C₁-C₄-alkoxycarbonyl, C₁-C₄-haloalkoxycarbonyl, N(R^{25a})R^{25b}, C(═O)NR^{25a}R^{25b}, Si(C₁-C₄-alkyl)₃;
C₁-C₄-alkyl, C₂-C₆-alkenyl, C₂-C₆-alkynyl, which are unsubstituted or partially or fully halogenated and/or substituted with CN, C₃-C₄-cycloalkyl, C₁-C₄-alkoxy, C₁-C₄-haloalkoxy, S(O)ₘ—C₁-C₄-alkyl, and oxo;
C₃-C₈-cycloalkyl which is unsubstituted or partially or fully halogenated and/or substituted with CN, C₁-C₄-alkyl, C₃-C₄-cycloalkyl, C₁-C₄-alkoxy, C₁-C₄-haloalkyl, C₁-C₄-haloalkoxy, S(O)ₘ—C₁-C₄-alkyl, and oxo; or
two R²²² present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl; or
two R²²² on two adjacent carbon atoms form together with the carbon atoms they are bonded to a 4-, 5-, 6-, 7-, or 8-membered saturated, partially unsaturated or maximally unsaturated ring, wherein the ring may contain 1 or 2 heteroatoms or heteroatom groups selected from N, O, and S(O)ₘ as ring members, and wherein the ring is optionally substituted with one or more groups C₁-C₄-alkyl, C₁-C₄-haloalkyl, C₁-C₄-alkoxy, and/or C₁-C₄-haloalkoxy;
m is 0, 1, or 2;
X is NR³ or O;
R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OR²¹, C₁-C₆-alkylthio, C₁-C₆-haloalkylthio, which are unsubstituted or partially or fully substituted with R³¹; or
C₁-C₆-alkyl-C(═O)OR²⁴, C₁-C₆-alkyl-C(=U)N(R^{25a})R^{25b}, C₁-C₆-alkyl-C(═NR²⁵)N(R^{25a})R^{25b}, C₁-C₆-alkyl-OC(═O)OR²⁴, N(R^{25a})R^{25b}, S(O)ₘR²⁴, S(O)ₘN(R^{25a})R^{25b}, C(=U)R²⁶, C(═O)OR²⁴, C(=U)N(R^{25a})R^{25b}, C(═S)SR²⁴, C(═NR²⁵)R²⁶;
C₃-C₈-cycloalkyl, C₃—C-halocycloalkyl, phenyl, a 3-, 4-, 5-, 6- or, 7-membered saturated or partially unsaturated heterocycle comprising 1, 2, 3, or 4 heteroatoms selected from N, O, and S(O)ₘ as ring members, or a 5- or 6-membered hetaryl comprising 1, 2, 3, or 4 heteroatoms selected from N, O, and S(O)ₘ as ring members, which rings are unsubstituted or partially or fully substituted with R³²;
R³¹ halogen, CN, NO₂, OH, SH, SCN, SF₅, Si(C₁-C₄-alkyl)₃, N(R^{25a})R^{25b}, C₁-C₆-alkoxy, C₁-C₆-haloalkoxy, C₁-C₆-alkyl-S(O)ₘ, C₁-C₆-haloalkyl-S(O)ₘ, C(═O)N(R^{25a})R^{25b};
C₃-C₈-cycloalkyl which is unsubstituted, partially or fully halogenated and/or partially or fully substituted with CN, C₁-C₄-alkyl, C₃-C₄-cycloalkyl, C₁-C₄-alkoxy, C₁-C₄-haloalkyl, C₁-C₄-haloalkoxy, and oxo; S(O)ₘR²⁴, S(O)ₘN(R^{25a})R^{25b}, C(=U)R²⁶, C(═O)OR²⁴, C(=U)N(R^{25a})R^{25b}, C(═S)SR²⁴, C(═NR²⁵)R²⁶;
phenyl, benzyl, phenoxy, or 3-, 4-, 5-, 6-, or 7-membered saturated, partially or fully unsaturated heterocycle containing 1, 2, or 3 heteroatoms selected from N, O, S(O)ₘ as ring members, wherein the rings are unsubstituted or partially or fully substituted with R²²²; or
two R³¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
R³² is a group as defined in R³¹ or selected from C₁-C₆-alkyl, C₂-C₆-alkenyl, and C₂-C₆-alkynyl, which groups are unsubstituted, partially or fully halogenated and/or substituted with one or two CN, C₃-C₄-cycloalkyl, C₁-C₄-alkoxy, C₁-C₄-haloalkoxy, or oxo;
G is phenyl, or 6-membered heteroaryl comprising as ring members 1 or 2 N-atoms; wherein G is unsubstituted, partially, or fully substituted with R²;
Q is an N-bound 5-membered heteroaryl comprising as ring members 1, 2, or 3 N-atoms; wherein Q is unsubstituted, partially, or fully substituted with R⁴;
R⁴ is halogen, NO₂, CN, OH, SH, SbF₅, C₁-C₆-alkoxy, C₁-C₆-haloalkoxy, C₁-C₄-alkylcarbonyl, C₁-C₄-haloalkylcarbonyl, C₁-C₄-alkoxycarbonyl, C₁-C₄-haloalkoxycarbonyl, Si(C₁-C₄-alkyl)₃; OR²¹, S(O)ₘ—C₁-C₄-alkyl, S(O)ₘ—C₁-C₄-haloalkyl, N(R^{25a})R^{25b}, S(O)ₘR²⁴, S(O)ₘN(R^{25a})R^{25b}, C(═NR²⁵)R²⁶, C(=U)R²⁶, C(═O)OR²⁴, C(=U)N(R^{25a})R^{25b}, C(═S)SR²⁴;
C₁-C₄-alkyl, C₂-C₆-alkenyl, C₂-C₆-alkynyl, which are unsubstituted or partially or fully halogenated and/or substituted with CN, C₃-C₆-cycloalkyl, C₁-C₄-alkoxy, C₁-C₄-haloalkoxy, S(O)ₘ—C₁-C₄-alkyl, S(O)ₘC₁-C₄-haloalkyl, N(R^{25a})R^{25b}, N(=O)(R^{25a})R^{25b}, and oxo;
C₃-C₆-cycloalkyl which is unsubstituted or partially or fully halogenated and/or substituted with CN, C₁-C₄-alkoxy, C₁-C₄-haloalkoxy, S(O)ₘ—C₁-C₄-alkyl, S(O)ₘ—C₁-C₄-alkyl, N(R^{25a})R^{25b}, N(=O)(R^{25a})R^{25b}; and oxo;
phenyl, benzyl, phenoxy, or 3-, 4-, 5-, 6-, or 7-membered saturated, partially or fully unsaturated heterocycle containing 1, 2, or 3 heteroatoms selected from N, O, S(O)ₘ as ring members, wherein the rings are unsubstituted or partially or fully substituted with R²²²;
and N-oxides, stereoisomers and agriculturally or veterinarily acceptable salts thereof.

2. The compound of formula I according to claim 1, wherein U is O.

3. The compound of formula I according to claim 1, wherein
W is phenyl, which is substituted with one to three groups halogen, halomethyl, halomethoxy, halomethyl-S(O)ₘ.

4. The compound of formula I according to claim 1, wherein
X is NR³, and
R³ is H, alkoxycarbonyl, C₁-C₆-alkyl, wherein alkyl is unsubstituted or substituted with halogen, CN, alkenyl, alkynyl, cycloalkyl, alkoxy.

5. The compound of formula I according to claim 1, wherein X is O.

6. The compound of formula I according to claim 1, wherein G is a group G1, G2, G3, or G4, 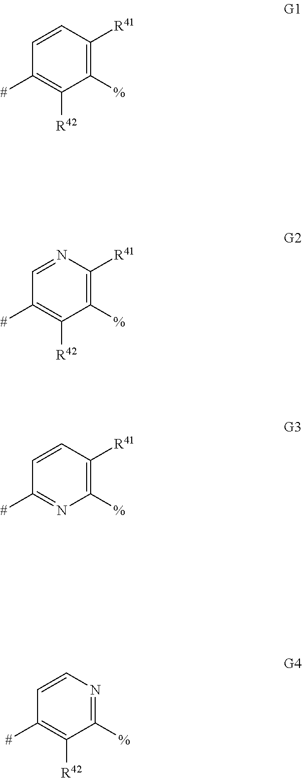
wherein # is the bond to X, % is the bond to Q, and R⁴¹ and R⁴² are H or a group R².

7. The compound of formula I according to claim 6, wherein G is G1 and R⁴² is H.

8. The compound of formula I according to claim 1, wherein R¹ is halomethyl.

9. The compound of formula I according to claim 1, wherein
R¹ is C₁-C₂-haloalkyl;
W is phenyl, which partially or fully substituted with R²;
R² is halogen, CN, OH, C₁-C₄-alkoxy, OR²¹, C₁-C₄-haloalkyl, C₁-C₃-alkoxy, C₁-C₃-haloalkoxy, C₁-C₃-alkyl-S(O)ₘ, C₁-C₃-haloalkyl-S(O)ₘ, C₁-C₃-alkoxy-C₁-C₄-alkyl;
X is NR³ or O;
R³ is H, C₁-C₄-alkyl, C₁-C₄-alkoxy-C₁-C₄-alkyl, C₁-C₄-alkoxycarbonyl, C₃-C₆-cycloalkyl-C₁-C₄-alkyl, C₂-C₄-alkenyl, C₂-C₄-alkynyl,
G is phenyl, which is unsubstituted, partially, or fully substituted with R²;
Q is pyrazole, imidazole, or triazole, Q being unsubstituted, or partially or fully substituted with CN, C₁-C₄-haloalkyl, C(═O)N(R^{12a})R^{12b}; wherein R^{12a}, R^{12b} are H, C₁-C₄-alkyl, C₁-C₄-haloalkyl.

10. The compound of formula I according to claim 1, which correspond to formula I.A

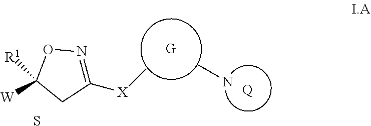

11. An agricultural or veterinary composition comprising at least one compound according to claim 1 and/or at least one agriculturally or veterinarily acceptable salt thereof, and at least one inert liquid and/or solid agriculturally or veterinarily acceptable carrier.

12. An agricultural composition for combating animal pests comprising at least one compound as defined in claim 1 and at least one inert liquid and/or solid acceptable carrier and, optionally, at least one surfactant.

13. A method for combating or controlling invertebrate pests, comprising contacting said invertebrate pests or their food supply, habitat or breeding grounds with a pesticidally effective amount of at least one compound as defined in claim 1.

14. A method for protecting growing plants from attack or infestation by invertebrate pests comprising contacting the plants, or soil or water in which the plants are growing, with a pesticidally effective amount of at least one compound as defined in claim 1.

15. A seed comprising a compound as defined in claim 1, or enantiomers, diastereomers, or salts thereof, in an amount of from 0.1 g to 10 kg per 100 kg of seed.

16. A method for treating or protecting an animal from infestation or infection by invertebrate pests comprising bringing the animal in contact with a pesticidally effective amount of at least one compound of the formula I as defined in claim 1, a stereoisomer thereof and/or at least one veterinarily acceptable salt thereof.
